SEQUENCE LISTING (98 sequences)

SEQ ID NO: 1 - protein, 501 aa, homo sapiens
MAQALPWLLLWMGAGVLPAHGTQHGIRLPLRSGLGGAPLGLRLPRETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDESTLMTIAYVMAAICALFMLPLCLMVCQWRCLRCLRQQHDDFADDISLLK

SEQ ID NO: 2 - protein, 10 aa, homo sapiens
SEVNLDAEFR

SEQ ID NO: 3 - protein, 16 aa, homo sapiens
TQHGIRLPLRSGLGGA

SEQ ID NO: 4 - protein, 6 aa, homo sapiens
LVPRGS

SEQ ID NO: 5 - protein, 7 aa, homo sapiens
  VARIANT 7: any amino acid
ENLYFNX

SEQ ID NO: 6 - protein, 7 aa, homo sapiens
PGAAHYA

SEQ ID NO: 7 - protein, 5 aa, homo sapiens
VRHQS

SEQ ID NO: 8 - protein, 28 aa, homo sapiens
GYYVEMTVGSPPQTLNILVDTGSSNFAV

SEQ ID NO: 9 - protein, 28 aa, homo sapiens
SSTYRDLRKGVYVPYTQGKWEGELGTDL

SEQ ID NO: 10 - protein, 21 aa, homo sapiens
ESDKFFINGSNWEGILGLAYA

SEQ ID NO: 11 - protein, 19 aa, homo sapiens
EYNYDKSIVDSGTTNLRLP

SEQ ID NO: 12 - protein, 12 aa, homo sapiens
  VARIANT 1: Glu, Met, Gln, Ser, Ala or Asp
XXXFVXMVDNLR

SEQ ID NO: 13 - protein, 10 aa, homo sapiens
SFVEMVDNLR

SEQ ID NO: 14 - protein, 10 aa, homo sapiens
QFVDMVDNLR

SEQ ID NO: 15 - protein, 12 aa, homo sapiens
SASFVEMVDNLR

SEQ ID NO: 16 - protein, 12 aa, homo sapiens
  VARIANT 5: any amino acid
LQLCXXXXGGSM

SEQ ID NO: 17 - protein, 12 aa, homo sapiens
  VARIANT 5: any amino acid
LRPVXXXXCYKF

SEQ ID NO: 18 - protein, 7 aa, homo sapiens
ELNLETD

SEQ ID NO: 19 - protein, 7 aa, homo sapiens
EINLETD

SEQ ID NO: 20 - protein, 7 aa, homo sapiens
EINFETD

SEQ ID NO: 21 - protein, 7 aa, homo sapiens
EVNLDAE

SEQ ID NO: 22 - protein, 8 aa, homo sapiens
EINFSFVE

SEQ ID NO: 23 - protein, 8 aa, homo sapiens
EINFQFVD

SEQ ID NO: 24 - protein, 8 aa, homo sapiens
EINFSASF

SEQ ID NO: 25 - protein, 6 aa, homo sapiens
EPGRRG

SEQ ID NO: 26 - protein, 24 aa, homo sapiens
GCTGCCCCGGGAGACCGACGAAGA

SEQ ID NO: 27 - protein, 24 aa, homo sapiens
CGGAGGTCCCGGTATGTGCTGGAC

SEQ ID NO: 28 - protein, 24 aa, homo sapiens
CCAGAGGCAGCTGTCCAGCACATA

SEQ ID NO: 29 - protein, 24 aa, homo sapiens
TCCCGCCGGATGGGTGTATACCAG

SEQ ID NO: 30 - protein, 27 aa, homo sapiens
GTACACAGGCAGTCTCTGGTATACACC

SEQ ID NO: 31 - protein, 29 aa, homo sapiens
GTGTGGTCCAGGGGAATCTCTATCTTCTG

SEQ ID NO: 32 - protein, 40 aa, homo sapiens
GTCATCGTCTCGAGTCACTTCAGCAGGGAGATGTCATCAG

SEQ ID NO: 33 - protein, 60 aa, homo sapiens
CGGCTGCCCCTGCGCAGCGGCCTGGGGGGCGCCCCCCTGGGGCTGCGGCTGCCCCGGGAG

SEQ ID NO: 34 - protein, 60 aa, homo sapiens
ATGGGCGCGGGAGTGCTGCCTGCCCACGGCACCCAGCACGGCATCCGGCTGCCCCTGCGC

SEQ ID NO: 35 - protein, 57 aa, homo sapiens
CCGGAATTCATGGCCCAAGCCCTGCCCTGGCTCCTGCTGTGGATGGGCGCGGGAGTG

SEQ ID NO: 36 - protein, 33 aa, homo sapiens
CGCCATATGGCGGGAGTGCTGCCTGCCCACGGC

SEQ ID NO: 37 - protein, 36 aa, homo sapiens
CCGGAATTCTCAGGTTGACTCATCTGTCTGTGGAAT

SEQ ID NO: 38 - protein, 439 aa, homo sapiens
TQHGIRLPLRSGLGGAPLGLRLPRLVPRGSETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDEST

SEQ ID NO: 39 - protein, 48 aa, homo sapiens
CTCTTCGTCGGTCTCAGAACCACGCGGAACCAGACGTGGCAGACGCAG

SEQ ID NO: 40 - protein, 433 aa, homo sapiens
TQHGIRLPLRSGLGGAPLGLRLPLETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDEST

SEQ ID NO: 41 - protein, 7 aa, homo sapiens
RLPLETD

SEQ ID NO: 42 - protein, 42 aa, homo sapiens
CTCTTCGTCGGTCTCCAGTGGCAGACGCAGACCCAGTGGAGC

SEQ ID NO: 43 - protein, 433 aa, homo sapiens
TQHGIRLPLRSGLGGAPLGLELNLETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDEST

SEQ ID NO: 44 - protein, 42 aa, homo sapiens
CTCTTCGTCGGTCTCCAGGTTCAGTTCCAGACCCAGTGGAGC

SEQ ID NO: 45 - protein, 27 aa, homo sapiens
GGTCTCCAGGTTGATTTCCAGACCCAG

SEQ ID NO: 46 - protein, 433 aa, homo sapiens
TQHGIRLPLRSGLGGAPLGLEINLETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDEST

SEQ ID NO: 47 - protein, 433 aa, homo sapiens
TQHGIRLPLRSGLGGAPLGLRLPRETDEEPEEPEINFSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDEST

SEQ ID NO: 48 - protein, 51 aa, homo sapiens
GACGAAGAGCCCGAGGAGCCCGAAATCAACTTCAGCTTTGTGGAGATGGTG

SEQ ID NO: 49 - protein, 51 aa, homo sapiens
CACCATCTCCACAAAGCTGAAGTTGATTTCGGGCTCCTCGGGCTCTTCGTC

SEQ ID NO: 50 - protein, 45 aa, homo sapiens
GAGCCCGAAATCAACTTCCAGTTTGTGGACATGGTGGACAACCTG

SEQ ID NO: 51 - protein, 45 aa, homo sapiens
CAGGTTGTCCACCATGTCCACAAACTGGAAGTTGATTTCGGGCTC

SEQ ID NO: 52 - protein, 45 aa, homo sapiens
CCCGAGGAGCCCGAAATCAACTTCTCCGCTAGCTTTGTGGAGATG

SEQ ID NO: 53 - protein, 45 aa, homo sapiens
CATCTCCACAAAGCTAGCGGAGAAGTTGATTTCGGGCTCCTCGGG

SEQ ID NO: 54 - protein, 433 aa, homo sapiens
TQHGIRLPLRSGLGGAPLGLRLPRETDEEPEEPEINFQFVDMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDEST

SEQ ID NO: 55 - protein, 435 aa, homo sapiens
TQHGIRLPLRSGLGGAPLGLRLPRETDEEPEEPEINFSASFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDEST

SEQ ID NO: 56 - protein, 48 aa, homo sapiens
GGTGCTCCACTGGGTCTGGAAATCAACCTGGAGACCGACGAAGAGCCC

SEQ ID NO: 57 - protein, 48 aa, homo sapiens
GGGCTCTTCGTCGGTCTCCAGGTTGATTTCCAGACCCAGTGGAGCACC

SEQ ID NO: 58 - protein, 433 aa, homo sapiens
TQHGIRLPLRSGLGGAPLGLEINLETDEEPEEPEINFSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDEST

SEQ ID NO: 59 - protein, 433 aa, homo sapiens
TQHGIRLPLRSGLGGAPLGLEINLETDEEPEEPEINFQFVDMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDEST

SEQ ID NO: 60 - protein, 435 aa, homo sapiens
TQHGIRLPLRSGLGGAPLGLEINLETDEEPEEPEINFSASFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDEST

SEQ ID NO: 61 - protein, 45 aa, homo sapiens
ATGGAAGACTGTGGCTACAACTGACCACAGACAGATGAGTCAACC

SEQ ID NO: 62 - protein, 45 aa, homo sapiens
GGTTGACTCATCTGTCTGTGGTCAGTTGTAGCCACAGTCTTCCAT

SEQ ID NO: 63 - protein, 27 aa, homo sapiens
GCCTGTATCCACACAGATGTTGAGCGT

SEQ ID NO: 64 - protein, 27 aa, homo sapiens
ACTGCTGCCTGTACACACCAGGATGTT

SEQ ID NO: 65 - protein, 27 aa, homo sapiens
CCACTTGCCCTGACAGTAGGGCACATA

SEQ ID NO: 66 - protein, 27 aa, homo sapiens
TTCCCACTTGCCACAGGTGTAGGGCAC

SEQ ID NO: 67 - protein, 27 aa, homo sapiens
GTTGATGAAGAAACAGTCTGATTCAGT

SEQ ID NO: 68 - protein, 27 aa, homo sapiens
GCCGTTGATGAAACACTTGTCTGATTC

SEQ ID NO: 69 - protein, 27 aa, homo sapiens
GTTGGAGCCGTTACAGAAGAACTTGTC

SEQ ID NO: 70 - protein, 27 aa, homo sapiens
CAGGATGCCTTCACAGTTGGAGCCGTT

SEQ ID NO: 71 - protein, 27 aa, homo sapiens
GGCCAGCCCCAGACAGCCTTCCCAGTT

SEQ ID NO: 72 - protein, 27 aa, homo sapiens
GTCAGGCCTGGCACACTCAGCATAGGC

SEQ ID NO: 73 - protein, 27 aa, homo sapiens
GGAGTCGTCAGGACAGGCAATCTCAGC

SEQ ID NO: 74 - protein, 27 aa, homo sapiens
GATCACCTCATAACACCACTCCCGCCG

SEQ ID NO: 75 - protein, 27 aa, homo sapiens
GTCCACAATGCTACAGTCATAGTTGTA

SEQ ID NO: 76 - protein, 27 aa, homo sapiens
GCCACTGTCCACACAGCTCTTGTCATA

SEQ ID NO: 77 - protein, 27 aa, homo sapiens
GGTGGTGCCACTACACACAATGCTCTT

SEQ ID NO: 78 - protein, 27 aa, homo sapiens
ACGAAGGTTGGTACAGCCACTGTCCAC

SEQ ID NO: 79 - protein, 27 aa, homo sapiens
GGGCAAACGAAGACAGGTGGTGCCACT

SEQ ID NO: 80 - protein, 27 aa, homo sapiens
TTTCTTGGGCAAACAAAGGTTGGTGGT

SEQ ID NO: 81 - protein, 27 aa, homo sapiens
CACCAGCTGCTCCACTAGCCAGAAACC

SEQ ID NO: 82 - protein, 27 aa, homo sapiens
ATCTTCCACTGGACACAGGTATTGCTG

SEQ ID NO: 83 - protein, 27 aa, homo sapiens
TGAGATGGCAAAACAGTAACAGTCGTC

SEQ ID NO: 84 - protein, 27 aa, homo sapiens
CGTGGATGACTGACAGATGGCAAACTT

SEQ ID NO: 85 - protein, 27 aa, homo sapiens
CATAACAGTGCCACAGGATGACTGTGA

SEQ ID NO: 86 - protein, 27 aa, homo sapiens
CAGCTCCCATACAAGTGCCCGTGGATG

SEQ ID NO: 87 - protein, 27 aa, homo sapiens
ACTGCTGCCTGTGTTCACCAGGATGTT

SEQ ID NO: 88 - protein, 27 aa, homo sapiens
GGTGGTGCCACTGTTCACAATGCTCTT

SEQ ID NO: 89 - protein, 39 aa, homo sapiens
AGCATTGTGGACAGTGGCTGCACCAACCTTCGTTTGCCC

SEQ ID NO: 90 - protein, 39 aa, homo sapiens
GGGCAAACGAAGGTTGGTGCAGCCACTGTCCACAATGCT

SEQ ID NO: 91 - protein, 47 aa, homo sapiens
GACAGTGGCACCACCAACCTTTGCTTGCCCAAGAAAGTGTTTGAAGC

SEQ ID NO: 92 - protein, 47 aa, homo sapiens
GCTTCAAACACTTTCTTGGGCAAGCAAAGGTTGGTGGTGCCACTGTC

SEQ ID NO: 93 - protein, 39 aa, homo sapiens
GCCATCTCACAGTCATCCTGCGGCACTGTTATGGGAGCT

SEQ ID NO: 94 - protein, 39 aa, homo sapiens
AGCTCCCATAACAGTGCCGCAGGATGACTGTGAGATGGC

SEQ ID NO: 95 - protein, 39 aa, homo sapiens
CAGTCATCCACGGGCACTTGCATGGGAGCTGTTATCATG

SEQ ID NO: 96 - protein, 39 aa, homo sapiens
CATGATAACAGCTCCCATGCAAGTGCCCGTGGATGACTG

SEQ ID NO: 97 - protein, 39 aa, homo sapiens
GAATCAGACAAGTTCTTCTGCAACGGCTCCAACTGGGAA

SEQ ID NO: 98 - protein, 39 aa, homo sapiens
TTCCCAGTTGGAGCCGTTGCAGAAGAACTTGTCTGATTC

(19) United States
(12) Patent Application Publication
(10) Pub. No.: US 2006/0019368 A1
(43) Pub. Date: Jan. 26, 2006

(54) Constructs for homogenously processed preparations of beta site APP-cleaving enzyme

(76) Inventors: Marcus Ballinger, Burlingame, CA (US); L. Michael Randal, Menlo Park, CA (US)

Correspondence Address: HELLER EHRMAN LLP, 275 MIDDLEFIELD ROAD, MENLO PARK, CA, 94025-3506, US

(21) Appl. No.: 10/895,861

(22) Filed: Jul. 20, 2004

(51) Int. Cl. C12N 9/64; C07H 21/04; C12P 21/06; C12N 15/74

(52) U.S. Cl. 435/226; 435/69.1; 435/320.1; 435/325; 536/23.2

(57) ABSTRACT

The present invention is directed to engineered polypeptides having BACE activity. In certain embodiments, the polypeptides also comprise an engineered cleavage site. Also provided are polypeptides comprising a prodomain, an engineered cleavage site, and a protease domain; the polypeptides are properly folded and are cleaved at the engineered cleavage site in vitro, producing homogeneous preparations of purified protease having BACE activity. The invention further pertains to nucleic acids, expression vectors, and host cells comprising the expression vectors for making the engineered polypeptides.

The present application claims priority to co-pending PCT Application Serial No. ______, filed on Jul. 7, 2004, which claims priority to U.S. patent application Ser. No. 10/726,967, filed Dec. 2, 2003; which claims priority to U.S. Provisional Patent Application No. 60/430,984, filed on Dec. 4, 2002 (now abandoned), the entire contents of which are incorporated herein by reference.

### BACKGROUND OF THE INVENTION

1. Field of the Invention

This invention provides constructs and polypeptides for producing preparations of homogeneously processed BACE.

2. Background of the Invention

β-secretases play an important role in disease. These proteins cleave the amyloid precursor protein (APP) at its β-secretase site; APP is subsequently cleaved at its γ-secretase site by γ-secretases, leading to the liberation of the Aβ peptide. Cerebral deposition of the Aβ peptide is part of the pathology of Alzheimer's disease and is also frequently observed in Down syndrome. Given the significance of β-secretases as disease targets, structural information obtained about their interaction with inhibitors is valuable in the design of compounds that inhibit β-secretase activity. An example of a β-secretase is beta site APP-cleaving enzyme, BACE1.

BACE1 is a transmembrane protein that operates in the lumen of the Golgi apparatus [Vassar R., et al., Science 286:735-741(1999)]. The full-length protein, also known as the preprosequence, comprises a presequence, a prodomain, a linker region, a protease domain, a transmembrane domain, and a cytosolic domain. The preproprotein sequence of the longest isoform of human BACE1, isoform A, is shown in SEQ ID NO:1. There are three other isoforms of BACE1—isoform B, isoform C, and isoform D—that lack residues 190-214, 146-189, and 146-214, respectively, relative to SEQ ID NO:1.

| SEQ ID NO:1 | |
|---|---|
| 1 | MAQALPWLLL WMGAGVLPAH GTQHGIRLPL RSGLGGAPLG LRLPRETDEE PEEPGRRGSF |
| 61 | VEMVDNLRGK SGQGYYVEMT VGSPPQTLNI LVDTGSSNFA VGAAPHPFLH RYYQRQLSST |
| 121 | YRDLRKGVYV PYTQGKWEGE LGTDLVSIPH GPNVTVRANI AAITESDKFF INGSNWEGIL |
| 181 | GLAYAEIARP DDSLEPFFDS LVKQTHVPNL FSLQLCGAGF PLNQSEVLAS VGGSMIIGGI |
| 241 | DHSLYTGSLW YTPIRREWYY EVIIVRVEIN GQDLKMDCKE YNYDKSIVDS GTTNLRLPKK |
| 301 | VFEAAVKSIK AASSTEKFPD GFWLGEQLVC WQAGTTPWNI FPVISLYLMG EVTNQSFRIT |
| 361 | ILPQQYLRPV EDVATSQDDC YKFAISQSST GTVMGAVIME GFYVVFDRAR KRIGFAVSAC |
| 421 | HVHDEERTAA VEGPFVTLDM EDCGYNIPQT DESTLMTIAY VMAAICALFM LPLCLMVCQW |
| 481 | RCLRCLRQQH DDFADDISLL K |

To date, there has been some degree of variability in the stated boundaries of some of the domains of BACE1; the amino acid numbering in the following is for isoform A. There appears to be consensus on the definitions of the presequence (1-21), the prodomain (22-45), and mature BACE1 (46-501). Boundaries of the transmembrane domain and the cytosolic domain have been within one to three amino acids of each other. For example, both SBASE and Swiss Prot have the potential transmembrane domain and the potential cytosolic domain consisting of residues 458-478 and 479-501, respectively. The boundaries for these two domains have also been described as 461-477, and 478-501, respectively [Shi, X.-P., et al., J Biol Chem 276:10366-10373 (2001)]. The protease domain has shown the most variability in its definition. It has alternately been placed between residues 46-460 [Shi, X.-P., et al., J Biol Chem 276:10366-10373 (2001)], 73-390 (S-BASE), and 74-416 (NP 036236). Additionally, a human BACE1 enzyme construct having an estimated protease domain ending at residue 419 showed activity against substrates [Lin, X. et al., Proc. Natl. Acad. Sci. USA 97:1456-1460 (2000)].

Other features of BACE1, isoform A, are catalytic Asp residues, D93 and D289, which are each part of a D(S/T)G motif; N-linked glycosylation sites at N153, N172, N223, and N354; and disulfide bonds formed between C216 and C420, between C278 and C443, and between C330 and C380 [Charlwood, J., et al., J Biol Chem 276:16739-16748 (2001)]. Additionally, mature BACE1 is phosphorylated at S498 in the cytosolic domain [Walter, J., et al., J Biol Chem 276:14634-14641 (2001)] and palmitoylated at residues C478, C482 and C485 in the cytosolic domain [Benjannet, S., et al., J. Biol. Chem. 276:10879-10887(2001)].

Soluble proteins containing the pre, pro, and protease domains can be expressed in bacterial and eukaryotic cell types [Vassar, R., et al., Science 286:735-741(1999); Lin, X., et al., Proc. Natl. Acad. Sci. USA 97:1456-1460 (2000); Mallender, W. D., et al., Mol Pharmacol. 59:619-626 (2001)]. The prodomain is required for folding in secreted expression systems [Shi, X.-P., et al., J Biol Chem 276:10366-10373 (2001)] and for refolding of protein expressed as inclusion bodies in E. coli. BACE expressed in Chinese hamster ovary cells is processed to yield an N-terminus at amino acid residue 46 (from the initiator Met residue), by a furin-like proprotein convertase (PC), which is believed to be responsible for the normal physiological processing in vivo [Benjannet, S., et al., J. Biol. Chem. 276:10879-10887 (2001); Creemers, J. W., et al., J. Biol. Chem. 276:4211-4217 (2001)]. Additionally, the substrate sequence preferences of purified BACE1 have been examined [See, for example, Turner, R. T. 3^{rd}, et al., Biochemistry 40:10001-10006 (2001); Gruninger-Leitch, F., et al., J. Biol. Chem. 277:4687-4693 (2002)].

Proteins produced by E. coli expression are generally preferred for X-ray crystallographic structure determination over those produced from insect or mammalian cells, because the former are devoid of heterogeneous glycosylation. However, previous attempts to express proBACE in E. coli and refold into active enzyme did not, however, generate crystals as a free enzyme or in complex with several small-molecule inhibitors. Hong, L., et al. [Science 290:150-153 (2000)] have determined the crystal structure of soluble BACE1 produced from E. coli, using protein that had been processed heterogeneously after amino acids Gly40 and Arg42-numbered relative to SEQ ID NO:1—presumably by a contaminating E. coli protease during purification. This processing event was not reproducible and would not be expected to be consistent from preparation to preparation. It is therefore useful to engineer proBACE constructs that can be processed homogeneously and robustly.

### SUMMARY OF THE INVENTION

This invention is directed to engineered polypeptides having BACE activity. The polypeptides are properly folded and are cleaved at an engineered cleavage site in vitro, producing homogeneous preparations of purified polypeptides having BACE activity.

The invention further pertains to nucleic acids, expression vectors, and host cells comprising the expression vectors for making the polypeptides having BACE activity.

### BRIEF DESCRIPTION OF THE DRAWING

FIG. 1 shows a deconvoluted mass spectrum of the processed product from the EINL-BACE1 polypeptide of SEQ ID NO:46. The apparent mass of the processed product is 45,558.0 Da, which is within 3.7 Da of the predicted mass of 45,561.7 Da for residues 25-433 of SEQ ID NO:46, corresponding to cleavage immediately C-terminal to the Leu24 of SEQ ID NO:46. Leu24 of SEQ ID NO:46 corresponds to Arg45 of the preprosequence (SEQ ID NO:1) of human BACE1.

### DEFINITIONS AND CERTAIN PREFERRED EMBODIMENTS OF THE INVENTION

“Acidic pH” as used herein refers to a pH less than 6, more preferably less than about 5.5, and most preferably between 4 and 5.5.

“BACE activity” is defined as the ability of a polypeptide under the in vitro conditions described by Mallender, W. D., et al., [Mol Pharmacol. 59:619-626 (2001)] to cleave the 29 amino acid peptide described therein comprising the Swedish mutant APP sequence SEVNLDAEFR (SEQ ID NO:2) at the scissile bond located between the Leu and the Asp contained within SEQ ID NO:2 with a specific activity of at least about 20 nmol/min/mg, preferably about 90 nmol/min/mg, more preferably about 200 nmol/min/mg, and even more preferably about 400 nmol/min/mg, and most preferably about 900 nmol/min/mg or higher.

The “prodomain” comprises at least six contiguous amino acids of SEQ ID NO:3. SEQ ID NO:3 corresponds to residues 22-37 of SEQ ID NO:1.

| | |
|---|---|
| TQHGTRLPLRSGLGGA | SEQ. ID NO: 3 |

In one embodiment, the prodomain comprises at least seven contiguous amino acids of SEQ ID NO:3. In another embodiment, the prodomain comprises at least eight contiguous amino acids of SEQ ID NO:3. In yet another embodiment, the prodomain comprises at least ten contiguous amino acids of SEQ ID NO:3. In still another embodiment, the prodomain comprises at least twelve contiguous amino acids of SEQ ID NO:3. In another embodiment, the prodomain comprises SEQ ID NO:3. In another embodiment, the prodomain comprises residues 22-41 of SEQ ID NO:1. In another embodiment, the prodomain comprises residues 22-45 of SEQ ID NO:1.

For purposes of shorthand designation of the polypeptides described herein, it is noted that numbers refer to the position of the altered amino acid residue along the amino acid sequences of respective wild-type protein sequence. Amino acid identification uses the single-letter alphabet of amino acids, i.e.

| | | |
|---|---|---|
| Asp | D | Aspartic acid |
| Ile | I | Isoleucine |
| Thr | T | Threonine |
| Leu | L | Leucine |
| Ser | S | Serine |
| Tyr | Y | Tyrosine |
| Glu | E | Glutamic acid |
| Phe | F | Phenylalanine |
| Pro | P | Proline |
| His | H | Histidine |
| Gly | G | Glycine |
| Lys | K | Lysine |
| Ala | A | Alanine |
| Arg | R | Arginine |
| Cys | C | Cysteine |
| Trp | W | Tryptophan |
| Val | V | Valine |
| Gln | Q | Glutamine |
| Met | M | Methionine |
| Asn | N | Asparagine |

Unless otherwise indicated, numbering of specific amino acids and amino acid sequences herein is with respect to the preprosequence of human BACE1 (SEQ ID NO:1).

Percent identity of a protein sequence to a reference protein sequence, is determined by alignment of the protein sequence to the reference protein sequence using the GAP program [Huang, X., Computer Applications in the Biosciences 10, 227-235 (1994)] in the Wisconsin Genetics Software Package Release 10.0, with a BLOSUM62 comparison matrix [Henikoff, S. & Henikoff, J. G., Proc Natl Acad Sci U S A 89:10915-10919 (1992)] and default parameters for gap openings and gap extensions. The alignment is calculated over the entire length of the reference sequence, with no penalty for gaps outside the alignment to the reference sequence. The program GAP is an implementation of the Needleman-Wunsch algorithm [Needleman & Wunsch, J. Mol. Biol., 48, 443-453 (1970)].

An “engineered cleavage site” as used herein is an autoproteolysis site or an exogenous protease cleavage site that has been introduced into a polypeptide having BACE activity.

An “autoproteolysis site” as used herein is defined as comprising the sequence X₁X₂X₃X₄X₅,
wherein
   X₁ is Glu or Gln;
   X₂ is Leu, Ile or Val,
   X₃ is Asn, Asp or Met,
   X₄ is Leu or Phe, and
   X₅ is Glu, Met, Gln, Ser, Ala or Asp.

An “exogenous protease” as used herein is a polypeptide having a protease activity other than BACE activity.

An “exogenous protease cleavage site” as used herein is a sequence comprising at least two amino acids that is cleaved by an exogenous protease. Examples of exogenous protease cleavage sites are a thrombin cleavage site, a tobacco etch virus cleavage site, a Genenase I cleavage site, an Enterokinase cleavage site, a Granzyme B cleavage site, a turnip mosaic virus protease NIa cleavage site, and a Factor Xa cleavage site.

A “thrombin cleavage site” as used herein is defined as the sequence PR, or as the sequence GRG, where in either case the scissile bond is located immediately after the Arg. More preferably it is the sequence LVPRGS (SEQ ID NO:4) where the scissile bond is located between the Arg and the Gly.

A “tobacco etch virus protease cleavage site”, used interchangeably herein with a “TEV protease cleavage site”, are each defined as the sequence ENLYFNX (SEQ ID NO:5), where X is any amino acid and wherein the scissile bond is located immediately after the second Asn. Preferably X is Gly or Ala.

A “Genenase I cleavage site” as used herein is defined as the sequence X₁X₂HX₃A, wherein X₁ is Ala, Phe or Tyr; X₂ is any amino acid; and X₃ is Phe, Leu or Tyr and wherein the scissile bond is located immediately before the last Ala of the sequence. More preferably it is the sequence PGAAHYA (SEQ ID NO:6) wherein the scissile bond is located immediately before the last Ala of the sequence.

An “Enterokinase cleavage site” as used herein refers to a sequence selected from the group consisting of DDK, DEK, EDK, and EEK, wherein for each case, the scissile bond is located immediately after the Lys; or a sequence selected from the group consisting of DDR, DER, EDR, and EER, wherein for each case, the scissile bond is located immediately after the Arg; and wherein the aforementioned sequences are not immediately followed by a Pro. More preferably, the enterokinase cleavage site is the sequence X₁X₂X₃X₄K, wherein X₁, X₂, X₃ and X₄ are each independently Asp or Glu, wherein the scissile bond is located immediately after the Lys, and wherein X₁X₂X₃X₄K is not immediately followed by a Pro.

A “Granzyme B cleavage site” as used herein refers to a sequence selected from the group consisting of IEXD, IMXD, IQXD, VEXD, VMXD, and VQXD, wherein for each case the scissile bond is located immediately after the Asp in the last position of the sequence. Preferably, the Granzyme B cleavage site is the sequence X₁X₂X₃DX₄X₅, wherein X₁ is Ile or Val, X₂ is Glu, Met or Gln, X₃ is any amino acid, X₄ is any amino acid, and X₅ is Gly or Ala, and wherein for each case the scissile bond is located immediately after the Asp in the fourth position of the sequence.

A “TuMV NIa protease cleavage site” used interchangeably herein with “turnip mosaic virus protease NIa cleavage site” is defined as the sequence VXHQ, wherein the scissile bond is located immediately after the Gln. More preferably, the TuMV NIa protease cleavage site is the sequence VRHQS (SEQ ID NO:7), wherein the scissile bond is located immediately after the Gln.

A “Factor Xa cleavage site” as used herein is defined as the sequence GR, wherein the scissile bond is located immediately after the Arg, and wherein the GR is not immediately followed by a Pro or an Arg. More preferably, the Factor Xa cleavage site is the sequence IXGR, wherein X is Asp or Glu, the scissile bond is located after the Arg, and IXGR is not immediately followed by a Pro or an Arg.

A “protease domain” as used herein is an amino acid sequence comprising at least 20 contiguous amino acids from residues 74-446 of SEQ ID NO:1. In one embodiment, the protease domain comprises at least 40 contiguous amino acids from residues 74-446 of SEQ ID NO:1. In another embodiment, the protease domain comprises at least 60 contiguous amino acids from residues 74-446 of SEQ ID NO:1. In another embodiment, the protease domain comprises at least 80 contiguous amino acids from residues 74-446 of SEQ ID NO:1. In another embodiment, the protease domain comprises at least 100 contiguous amino acids from residues 74-446 of SEQ ID NO:1. In another embodiment, the protease domain comprises at least 120 contiguous amino acids from residues 74-446 of SEQ ID NO:1. In another embodiment, the protease domain comprises at least 180 contiguous amino acids from residues 74-446 of SEQ ID NO:1. In yet another embodiment, the protease domain comprises at least 240 contiguous amino acids from residues 74-446 of SEQ ID NO:1. In still another embodiment, the protease domain comprises at least 300 contiguous amino acid from residues 74-446 of SEQ ID NO:1.

In another embodiment, the protease domain comprises at least one sequence selected from the group consisting of SEQ ID NO:8, SEQ ID NO:9, SEQ ID NO:10 and SEQ ID NO:11.

| | |
|---|---|
| GYYVEMTVGSPPQTLNILVDTGSSNFAV | SEQ ID NO:8 |
| SSTYRDLRKGVYVPYTQGKWEGELGTDL | SEQ ID NO:9 |
| ESDKFFINGSNWEGILGLAYA | SEQ ID NO:10 |
| EYNYDKSIVDSGTTNLRLP | SEQ ID NO:11 |

SEQ ID NO:8 corresponds to residues 74-101 of isoforms A, B, C, and D of human BACE1, and SEQ ID NO:9 corresponds to residues 118-145 of isoforms A, B, C, and D of human BACE1. SEQ ID NO:10 corresponds to residues 165-185 of human BACE1 isoform A and human BACE1 isoform B, and is not present in human BACE1 isoform C or human BACE1 isoform D. SEQ ID NO:11 corresponds to residues 280-298 of human BACE1 isoform A, residues 255-273 of human BACE1 isoform B, residues 236-254 of human BACE1 isoform C, and residues 211-229 of human BACE1 isoform D, respectively. In one embodiment, the protease domain comprises SEQ ID NO:8. In another embodiment, the protease domain comprises SEQ ID NO:9. In another embodiment, the protease domain comprises both SEQ ID NO:8 and SEQ ID NO:9. In another embodiment, the protease domain comprises SEQ ID NO:8, SEQ ID NO:9, or both SEQ ID NO:8 and SEQ 11) NO:9, and further comprises at least one sequence selected from the group consisting of SEQ ID NO:10 and SEQ ID NO:11. In yet another embodiment, the protease domain comprises SEQ ID NO:8 and SEQ ID NO:10. In another embodiment the protease domain comprises SEQ ID NO:8 and SEQ ID NO:11. In another embodiment, the protease domain comprises SEQ ID NO:9 and SEQ ID NO:10. In another embodiment, the protease domain comprises SEQ ID NO:9 and SEQ ID NO:11. In another embodiment, the protease domain comprises SEQ ID NO:8, SEQ ID NO:9 and SEQ ID NO:10. In another embodiment, the protease domain comprises SEQ ID NO:8, SEQ ID NO:9 and SEQ ID NO:11. In another embodiment, the protease domain comprises SEQ ID NO:8, SEQ ID NO:10 and SEQ ID NO:11. In another embodiment, the protease domain comprises SEQ ID NO:9, SEQ ID NO:10, and SEQ ID NO:1. In still another embodiment, the protease domain comprises SEQ ID NO:8, SEQ ID NO:9, SEQ ID NO:10 and SEQ ID NO:11.

The invention relates to engineered polypeptides having BACE activity. In certain embodiments, the polypeptides have at least one engineered cleavage site allowing proteolysis or autoproteolysis of the polypeptide in vitro. Such polypeptides comprise in order from N-terminus to C-terminus: a prodomain; an engineered cleavage site; and a protease domain; wherein the polypeptide is capable of being cleaved at the engineered cleavage site thereby releasing a free protease domain that has BACE activity. In other embodiments, the invention is directed to novel polypeptides having BACE activity corresponding to a free protease domain released via autoproteolysis at acidic pH.

In still other embodiments, polypeptides of the invention have one or more features designed to promote crystallization of BACE. For example, some polypeptides of the invention are soluble. The term “soluble” as used in reference to the polypeptides herein, is defined as lacking transmembrane and cytosolic domains. In another example, certain polypeptides lack flexible regions near the C-terminus of the protease domain. In yet another example, certain polypeptides lack one or more flexible regions within the protease domain.

Processed BACE

In one aspect, the invention is directed to a polypeptide comprising a protease domain, wherein the N-terminal sequence of the polypeptide is X₁X₂X₃FVX₄MVDNLR (SEQ ID NO:12),
wherein X₁ and X₄ are each independently Glu, Met, Gln, Ser, Ala or Asp; and X₂ and X₃ are each independently absent, Glu, Met, Gln, Ser, Ala or Asp; and wherein the polypeptide comprises BACE activity.

In one embodiment, the N-terminal sequence of the polypeptide is selected from the group consisting of SFVEMVDNLR (SEQ ID NO:13), QFVDMVDNLR (SEQ ID NO:14) and SASFVEMVDNLR (SEQ ID NO:15).

In another embodiment of the polypeptide, the protease domain comprises at least 40 contiguous amino acids from residues 74-446 of SEQ ID NO:1. In another embodiment of the polypeptide, the protease domain comprises at least 60 contiguous amino acids from residues 74-446 of SEQ ID NO:1.

In another embodiment of the polypeptide, the protease domain comprises at least one sequence selected from the group consisting of SEQ ID NO:8, SEQ ID NO:9, SEQ ID NO:10 and SEQ ID NO:11.

In yet another embodiment, the polypeptide has at its C-terminus a sequence corresponding to residues 447-454 of SEQ ID NO:1. Alternatively, in a different embodiment, the polypeptide has at its C-terminus a sequence corresponding to residues 441-446 of SEQ ID NO:1.

In still another embodiment, the polypeptide consists of a sequence at least 95% identical to residues 59-446 of SEQ ID NO:1. In another embodiment, the polypeptide consists of a sequence at least 95% identical to residues 59-446 of SEQ ID NO:1 and has at its C-terminus a sequence corresponding to residues 447-454 of SEQ ID NO:1. Alternatively, in a different embodiment, the polypeptide consists of a sequence at least 95% identical to residues 59-446 of SEQ ID NO:1 and has at its C-terminus a sequence corresponding to residues 441-446 of SEQ ID NO:1.

Engineered Loops

In other polypeptides of the invention, a polypeptide having BACE activity is engineered to have one or more altered loops within the protease domain. The polypeptides having altered loops may or may not comprise an engineered cleavage site. Preferably, the altered loops are shortened as compared with those of the wild-type enzyme, so as to decrease the flexibility of the polypeptide and thereby promote crystallization. Such polypeptides preferably contain one or more sequences that have similarity to certain regions of BACE that are more ordered; these regions correspond to, e.g., residues 74-207 of SEQ ID NO:1, residues 241-361 of SEQ ID NO:1 and residues 389-446 of SEQ ID NO:1. Furthermore, the polypeptides typically do not include one or more sequences of BACE that form long and/or disordered loops, referred to herein as loop 1 and loop 2. Loop 1 corresponds to residues 217-231 of SEQ ID NO:1; loop 2 corresponds to residues 371-379 of SEQ ID NO:1. Thus it is preferable that loop 1 and/or loop 2 sequences are replaced with sequences that are shorter in length and that promote formation of a hairpin turn.

Certain polypeptides with shortened loops contain at least one sequence selected from LQLCX₁X₂X₃X₄GGSM (SEQ ID NO:16) and LRPVX₁X₂X₃X₄CYKF (SEQ ID NO:17) wherein each instance of X₁ and X₂ is independently any amino acid and wherein each instance of X₃ and X₄ is independently absent or any amino acid. Specifically defined residues in SEQ ID NO:16 correspond to regions flanking loop 1; specifically defined residues in SEQ ID NO:17 correspond to regions flanking loop 2. In particular, the first four residues of SEQ ID NO:16 correspond to residues 213-216 of SEQ ID NO:1; the last four residues of SEQ ID NO:16 correspond to residues 232-235 of SEQ ID NO:1. The first four residues of SEQ ID NO:17 correspond to residues 367-370 of SEQ ID NO:1; the last four residues of SEQ ID NO:17 correspond to residues 380-383 of SEQ ID NO:1. Thus the X₁X₂X₃X₄ sequence intervening between the loop flanking sequences present in each of SEQ ID NO:16 and SEQ ID NO:17 corresponds to a loop replacement sequence that is 2, 3 or 4 residues in length. Preferably X₁ and X₂, and X₃ and X₄ if present, are residues that allow formation of a stable hairpin turn. For example, for SEQ ID NO:16 and for SEQ ID NO:17, each instance of X₁ and X₂, and each instance of X₃ and X₄ if present, may be a serine.

Thus in another aspect, the invention is directed to a polypeptide comprising a sequence at least 85% identical to residues 74-446 of SEQ ID NO:1, wherein the polypeptide does not include at least one sequence selected from the group consisting of a sequence corresponding to residues 217-231 of SEQ ID NO:1 and sequence corresponding to residues 371-379 of SEQ ID NO:1.

In one embodiment, the polypeptide comprises a sequence at least 90% identical to residues 74-446 of SEQ ID NO:1.

In another embodiment, the polypeptide comprises a sequence at least 85% identical to residues 74-446 of SEQ ID NO:1, and does not include a sequence corresponding to residues 217-231 of SEQ ID NO:1 and a sequence corresponding to residues 371-379 of SEQ ID NO:1.

In another embodiment, the polypeptide comprises a sequence at least 85% identical to residues 74-446 of SEQ ID NO:1, wherein the polypeptide does not include at least one sequence selected from the group consisting of a sequence corresponding to residues 217-231 of SEQ ID NO:1 and a sequence corresponding to residues 371-379 of SEQ ID NO:1; and wherein the polypeptide comprises at least one sequence selected from the group consisting of LQLCX₁X₂X₃X₄GGSM (SEQ ID NO:16) and LRPVX₁X₂X₃X₄CYKF (SEQ ID NO:17) wherein each instance of X₁ and X₂ is independently any amino acid and wherein each instance of X₃ and X₄ is independently absent or any amino acid.

In another embodiment, the polypeptide does not include a sequence corresponding to residues 217-231 of SEQ ID NO:1, and the polypeptide comprises SEQ ID NO:16 as defined herein. In another embodiment, the polypeptide does not include a sequence corresponding to residues 371-379 of SEQ ID NO:1, and the polypeptide comprises SEQ ID NO:17 as defined herein. In another embodiment, the polypeptide does not include a sequence corresponding to residues 217-231 of SEQ ID NO:1 and a sequence corresponding to residues 371-379 of SEQ ID NO:1, and the polypeptide comprises both SEQ ID NO:16 and SEQ ID NO:17 each as defined herein.

In another embodiment, the polypeptide comprises at least one sequence selected from the group consisting of SEQ ID NO:8, SEQ ID NO:9, SEQ ID NO:10 and SEQ ID NO:11.

In yet another embodiment, the polypeptide comprises an autoproteolysis site as defined herein.

Engineered proBACE

In some polypeptides of the invention, an engineered cleavage site is introduced into proBACE to facilitate folding and enable production of homogenous preparations of processed BACE. Thus in another aspect, the invention is directed to a polypeptide comprising in order from N-terminus to C-terminus:
a prodomain comprising at least six contiguous amino acids of SEQ ID NO:3;
an engineered cleavage site; and
a protease domain;
   wherein the polypeptide is capable of being cleaved at the engineered cleavage site thereby releasing a free protease domain that has BACE activity.

In one embodiment of the polypeptide, the prodomain comprises at least seven contiguous amino acids of SEQ ID NO:3. In another embodiment, the prodomain comprises at least eight contiguous amino acids of SEQ ID NO:3. In yet another embodiment, the prodomain comprises at least ten contiguous amino acids of SEQ ID NO:3. In still another embodiment, the prodomain comprises at least twelve contiguous amino acids of SEQ ID NO:3. In a preferred embodiment, the prodomain comprises SEQ ID NO:3.

In another embodiment of the polypeptide, the engineered cleavage site is an autoproteolysis site, as defined herein. In another embodiment of the polypeptide, the engineered cleavage site is an exogenous protease cleavage site, as defined herein.

Autoproteolysis Sites

In certain polypeptides of this invention, a polypeptide having BACE activity is engineered for autoproteolysis. Autoproteolysis sites for BACE1 as used herein comprise the amino acid sequence X₁X₂X₃X₄X₅, where X₁, X₂, X₃, X₄, and X₅ generally correspond to substrate sequence positions P4, P3, P2, P1, and P1′, respectively, according to the nomenclature of Schechter and Berger [Schechter, I. & Berger, A., Biochem. Biophys. Res. Commun. 27:157-162 (1967)].

A particular aspect of the invention is a polypeptide comprising in order from N-terminus to C-terminus:
a) a prodomain comprising SEQ ID NO:3;
b) an autoproteolysis site comprising the sequence X₁X₂X₃X₄X₅,
   wherein
      X₁ is Glu or Gln,
      X₂ is Leu, Ile or Val,
      X₃ is Asn, Asp or Met,
      X₄ is Leu or Phe, and
      X₅ is Glu, Met, Gln, Ser, Ala or Asp; and
c) a protease domain comprising at least 20 contiguous amino acids from residues 74-446 of SEQ ID NO:1;
wherein the polypeptide is capable of being cleaved at the autoproteolysis site thereby releasing a free protease domain that has BACE activity.

In one embodiment of the polypeptide, the autoproteolysis site comprises the sequence X₁X₂X₃X₄X₅,
wherein
   X₁ is Glu,
   X₂ is Leu, Ile or Val,
   X₃ is Asn,
   X₄ is Leu or Phe, and
   X₅ is Glu, Gln, Ser, or Asp.

In another embodiment of the polypeptide, the autoproteolysis site is selected from the group consisting of:

| | |
|---|---|
| a) ELNLETD | (SEQ ID NO:18), |
| b) EINLETD | (SEQ ID NO:19), |
| c) EINFETD | (SEQ ID NO:20), |
| and | |
| d) EVNLDAE | (SEQ ID NO:21). |

In yet another embodiment of the polypeptide, the autoproteolysis site is selected from the group consisting of:

| | |
|---|---|
| a) EINFSFVE | (SEQ ID NO:22), |
| b) ETNEQFVD | (SEQ ID NO:23), |
| and | |
| c) ETNFSASF | (SEQ ID NO:24). |

In still another embodiment of the polypeptide, the prodomain comprises residues 22-41 of SEQ ID NO:1. In another embodiment of the polypeptide, the prodomain comprises residues 22-45 of SEQ ID NO:1.

In another embodiment of the polypeptide, the protease domain comprises at least 40 contiguous amino acids from residues 74-446 of SEQ ID NO:1. In another embodiment of the polypeptide, the protease domain comprises at least 60 contiguous amino acids from residues 74-446 of SEQ ID NO:1. In another embodiment of the polypeptide, the protease domain comprises at least 80 contiguous amino acids from residues 74-446 of SEQ ID NO:1. In another embodiment of the polypeptide, the protease domain comprises at least 100 contiguous amino acids from residues 74-446 of SEQ ID NO:1. In another embodiment of the polypeptide, the protease domain comprises at least 120 contiguous amino acids from residues 74-446 of SEQ ID NO:1. In another embodiment of the polypeptide, the protease domain comprises at least 180 contiguous amino acids from residues 74-446 of SEQ ID NO:1. In another embodiment of the polypeptide, the protease domain comprises at least 240 contiguous amino acids from residues 74-446 of SEQ ID NO:1. In another embodiment of the polypeptide, the protease domain comprises at least 300 contiguous amino acids from residues 74-446 of SEQ ID NO:1. In another embodiment of the polypeptide, the protease domain comprises residues 74-446 of SEQ ID NO:1.

In another embodiment of the polypeptide, the protease domain comprises at least one sequence selected from the group consisting of SEQ ID NO:8, SEQ ID NO:9, SEQ ID NO: 10 and SEQ ID NO:11.

In another aspect, the invention is directed to a polypeptide comprising an amino acid sequence that is at least 85% identical to residues 22-446 of SEQ ID NO:1 wherein the sequence includes an autoproteolysis site selected from the group consisting of ELNLETD (SEQ ID NO:18), EINLETD (SEQ ID NO:19), EINFETD (SEQ ID NO:20), EVNLDAE (SEQ ID NO:21), EINFSFVE (SEQ ID NO:22), EINFQFVD (SEQ ID NO:23) and EINFSASF (SEQ ID NO:24).

In one embodiment, the polypeptide has at its C-terminus a sequence corresponding to residues 447-454 of SEQ ID NO:1. Alternatively, in a different embodiment, the polypeptide has at its C-terminus a sequence corresponding to residues 441-446 of SEQ ID NO:1.

In another embodiment of the polypeptide, the sequence is at least 90% identical to residues 22-446 of SEQ ID NO:1. In another embodiment of the polypeptide, the sequence is at least 95% identical to residues 22-446 of SEQ ID NO:1. In another embodiment of the polypeptide, the sequence is at least 97% identical to residues 22-446 of SEQ ID NO:1. In another embodiment of the polypeptide, the sequence is at least 99% identical to residues 22-446 of SEQ ID NO:1.

In another aspect, the invention is directed to a polypeptide comprising in order from N-terminus to C-terminus:
(i) a prodomain comprising at least six contiguous amino acids of SEQ ID NO:3;
(ii) an autoproteolysis site comprising the sequence X₁X₂X₃X₄X₅.
wherein
   X₁ is Glu or Gln,
   X₂ is Leu, Ile or Val,
   X₃ is Asn, Asp or Met,
   X₄ is Leu or Phe, and
   X₅ is Glu, Met, Gln, Ser, Ala or Asp; and
(iii) a protease domain comprising at least one amino acid sequence selected from the group consisting of
   (a) a sequence at least 90% identical to residues 74-207 of SEQ ID NO:1,
   (b) a sequence at least 90% identical to residues 241-361 of SEQ ID NO:1 and
   (c) a sequence at least 90% identical to residues 389-446 of SEQ ID NO:1;
wherein the polypeptide is capable of being cleaved at the autoproteolysis site thereby releasing a free protease domain that has BACE activity.

In one embodiment, the polypeptide has at its C-terminus the sequence corresponding to residues 447-454 of SEQ ID NO:1. Alternatively, in a different embodiment, the polypeptide has at its C-terminus a sequence corresponding to residues 441-446 of SEQ ID NO:1.

In another embodiment of the polypeptide, the autoproteolysis site comprises the sequence X₁X₂X₃X₄X₅,

wherein

X₁ is Glu,
X₂ is Leu, Ile or Val,
X₃ is Asn,
X₄ is Leu or Phe, and
X₅ is Glu, Gln, Ser, or Asp.

In still another embodiment of the polypeptide, the autoproteolysis site is selected from the group consisting of ELNLETD (SEQ ID NO:18), EINLETD (SEQ ID NO:19), EINFETD (SEQ ID NO:20), EVNLDAE (SEQ ID NO:21), EINFSFVE (SEQ ID NO:22), EINFQFVD (SEQ ID NO:23) and EINFSASF (SEQ ID NO:24).

In another embodiment of the polypeptide, the protease domain comprises at least two sequences selected from (a), (b) and (c). In another embodiment of the polypeptide, the protease domain comprises each of (a), (b) and (c).

In yet another embodiment of the polypeptide, the protease domain comprises at least one sequence selected from the group consisting of a sequence at least 97% identical to residues 74-207 of SEQ ID NO:1; a sequence at least 97% identical to residues 241-361 of SEQ ID NO:1; and a sequence at least 97% identical to residues 389-446 of SEQ ID NO:1.

In another embodiment, the polypeptide comprises BACE activity and comprises in order from N-terminus to C-terminus:
(i) a prodomain comprising at least six contiguous amino acids of SEQ ID NO:3;
(ii) an autoproteolysis site comprising the sequence X₁X₂X₃X₄X₅,
wherein
   X₁ is Glu or Gln,
   X₂ is Leu, Ile or Val,
   X₃ is Asn, Asp or Met,
   X₄ is Leu or Phe, and
   X₅ is Glu, Met, Gln, Ser, Ala or Asp; and
(iii) a protease domain comprising at least one amino acid sequence selected from the group consisting of
   (a) a sequence at least 90% identical to residues 74-207 of SEQ ID NO:1,
   (b) a sequence at least 90% identical to residues 241-361 of SEQ ID NO:1 and
   (c) a sequence at least 90% identical to residues 389-446 of SEQ ID NO:1;
wherein the polypeptide is capable of being cleaved at the autoproteolysis site thereby releasing a free protease domain that has BACE activity; and wherein the protease domain does not include at least one sequence selected from the group consisting of a sequence corresponding to residues 217-231 of SEQ ID NO:1 and a sequence corresponding to residues 371-379 of SEQ ID NO:1.

In yet another embodiment, the protease domain does not include the sequence corresponding to residues 217-231 of SEQ ID NO:1 and the sequence corresponding to residues 371-379 of SEQ ID NO:1.

In another embodiment, the polypeptide comprises BACE activity and comprises in order from N-terminus to C-terminus:
(i) a prodomain comprising at least six contiguous amino acids of SEQ ID NO:3;
(ii) an autoproteolysis site comprising the sequence X₁X₂X₃X₄X₅,
wherein
   X₁ is Glu or Gln,
   X₂ is Leu, Ile or Val,
   X₃ is Asn, Asp or Met,
   X₄ is Leu or Phe, and
   X₅ is Glu, Met, Gln, Ser, Ala or Asp; and
(iii) a protease domain comprising at least one amino acid sequence selected from the group consisting of
   (a) a sequence at least 90% identical to residues 74-207 of SEQ ID NO:1,
   (b) a sequence at least 90% identical to residues 241-361 of SEQ ID NO:1 and
   (c) a sequence at least 90% identical to residues 389-446 of SEQ ID NO:1, and
where the protease domain comprises at least one sequence selected from the group consisting of
   (d) LQLCX₁X₂X₃X₄GGSM (SEQ ID NO:16) and
   (e) LRPVX₁X₂X₃X₄CYKF (SEQ ID NO:17),
wherein each instance of X₁ and X₂ is independently any amino acid and
wherein each instance of X₃ and X₄ is independently absent or any amino acid.
In another embodiment, the protease domain comprises both
(d) LQLCX₁X₂X₃X₄GGSM (SEQ ID NO:16) and
(e) LRPVX₁X₂X₃X₄CYKF (SEQ ID NO:17),
   wherein each instance of X₁ and X₂ is independently any amino acid and
   wherein each instance of X₃ and X₄ is independently absent or any amino acid.
      Exogenous Protease Cleavage

In other polypeptides of the invention, a polypeptide having BACE activity is engineered for exogenous protease cleavage. An exogenous protease cleavage site is a sequence comprising at least two amino acids which sequence is cleaved by an exogenous protease, a polypeptide having protease activity other than BACE activity.

In one embodiment, the invention is directed to a polypeptide comprising in order from N-terminus to C-terminus:
a) a prodomain comprising at least six contiguous amino acids of SEQ ID NO:3;
b) an exogenous protease cleavage site; and
c) a protease domain;
   wherein the polypeptide is capable of being cleaved at the exogenous protease cleavage site thereby releasing a free protease domain that has BACE activity. In a particular embodiment, the exogenous protease cleavage site is a thrombin cleavage site, a tobacco etch virus cleavage site, a Genenase I cleavage site, an Enterokinase cleavage site, a Granzyme B cleavage site, a turnip mosaic virus protease NIa cleavage site, or a Factor Xa cleavage site.

Conditions for the in vitro cleavage of the engineered cleavage site by the exogenous protease should be chosen so as to optimize the selectivity of the exogenous protease for its preferred cleavage sites over any other potential or non-preferred sites. Effects of adjustment of certain cleavage conditions are well known in the art. For example, greater specificity is achieved in enzymatic reactions having a low enzyme: substrate ratio. Optimally, the lowest amount of enzyme necessary for complete and specific cleavage should be used. Additionally, enzymes can show greater specificity in substrate cleavage at lower temperatures, for example, at 4° C. relative to at 37° C.

Engineered Cleavage Sites

An important consideration in designing the engineered cleavage site is the selectivity of the protease to be used with respect to the sequence of the remainder of the polypeptide having BACE activity, especially of its protease domain. For example, in particular embodiments of this invention, exogenous protease cleavage sites of enzymes having well-characterized selectivity are preferred. Furthermore, it is preferable that the engineered cleavage site is created such that there are few preferred or non-preferred sequences, preferably less than three, that may be cleaved by the exogenous protease or by autoproteolysis occurring downstream of the engineered cleavage site within the polypeptide. It is possible that uniform processing could occur in a polypeptide having BACE activity that contains more than one cleavage site for a given protease, particularly if the engineered cleavage site selected were highly preferred by that protease over other existing sites. Furthermore, it is possible that uniform processing could be effected by a protease where equally preferred sites exist, because only the engineered cleavage site may be accessible to the protease when the polypeptide having BACE activity exists in a folded state. Additionally, it is allowable that additional, non-overlapping sites for the protease to be present upstream (i.e., on the N-terminal side) of the engineered cleavage site in the polypeptide, as long as the engineered cleavage site is more preferred than the additional upstream sites.

In particular, is preferable to place the engineered cleavage sites within the polypeptide having BACE activity such that there are not overlapping sites spaced closely together, i.e., within less than about 5 amino acids of one another. Cleavage sites that are placed closely enough to overlap may have similar rates of cleavage. For overlapping sites located closer to the N-terminus of the protein, cleavage at the N-terminal cleavage site would prevent cleavage at the C-terminal cleavage site because upon cleavage of the N-terminal site, a portion of the C-terminal site would be removed. Thus overlapping engineered cleavage sites are expected to produce heterogeneous cleavage products.

In a preferred embodiment of polypeptides having BACE activity and comprising an engineered cleavage site, the polypeptide has no sites occurring within the protease domain that can be cleaved by the corresponding exogenous protease or by autoproteolysis. An exogenous protease cleavage site or autoproteolysis site located within the protease domain is disadvantageous as cleavage at that site may reduce or eliminate the BACE activity of the released product. Exogenous protease cleavage sites or autoproteolysis sites contained within the protease domain may be eliminated through alteration of the protease domain itself so that the site contained therein is removed while BACE activity is retained.

Specific examples of preferred exogenous protease cleavage sites not occurring in the protease domain of native BACE1 include the TEV protease cleavage site, the Genenase I cleavage site, the Enterokinase cleavage site, a Granzyme B cleavage site, and the TuMV protease NIa cleavage site. Another example of a preferred engineered cleavage site is a Factor Xa cleavage site. Although it would appear that, for example, BACE1 has a Factor Xa cleavage site at ⁵³EPGRRG⁵⁸ (SEQ ID NO:25), with cleavage occurring after Arg56, the P4 and P3 positions of this site do not conform to a preferred Factor Xa cleavage site. Moreover, it is also known that Factor Xa will not cleave a site followed by Pro or Arg. Examples of more preferred exogenous protease cleavage sites-expected to be targets of highly specific cleavage in the context of BACE1—are preferred TEV protease cleavage sites, preferred Enterokinase cleavage sites, preferred TuMV protease NIa cleavage sites, the preferred Genenase I cleavage site, or preferred Factor Xa cleavage sites.

It is possible to construct polypeptides having BACE activity and comprising engineered cleavage sites by modification of a native BACE1 sequence. For example, one or more amino acids may be introduced between the N-terminal residue of the prodomain, and the protease domain. In particular, an engineered cleavage site may be composed entirely of a stretch of residues inserted between two residues of this region, for example, see SEQ ID NO:38 (soluble human BACE1 with a preferred thrombin site insert). Alternatively, one or more amino acids may be substituted with different amino acids, e.g., see SEQ ID NO:43. If more than one amino acid is substituted, the amino acids having substitutions may be contiguous or noncontiguous. Another means of introducing the engineered cleavage site involves deletion of one or more amino acids, so that the remaining sequence surrounding the deleted residue or residues constitutes the engineered cleavage site. Finally, any combination of insertion, substitution or deletion of amino acids may be used to introduce the engineered cleavage site at the desired position of BACE1.

Cysteine Mutants

In another aspect, the invention is directed to a polypeptide comprising BACE activity and further comprising a substitution to a cysteine of a native noncysteine residue. Such polypeptides are useful for discovery and development of BACE1 inhibitors by use of Tethering^{SM}, which is described in U.S. Pat. No. 6,335,155, and PCT publications WO 02/42773, and WO 03/046200. In brief, Tethering^{SM} enables discovery of low molecular weight compounds that weakly bind to a region on a protein target, through formation of a covalent bond between the compound and, for example, a cysteine residue located at the region. Thus a polypeptide having BACE activity and comprising an introduced cysteine may be used to screen against a library of compounds each containing a group capable of reacting with the cysteine thiol. Under certain conditions, the subset of compounds having noncovalent binding affinity to the region at which the cysteine is located can form a covalent linkage to the introduced cysteine on the polypeptide. The resulting polypeptide-compound conjugate may subsequently be identified, for example, by mass spectroscopy. The protease domain of native human BACE1 comprises six cysteines, each of which are involved in disulfide bond formation. Therefore, it is preferable that polypeptides used to find compounds binding the protease domain by Tethering^{SM} comprise an additional, reactive cysteine located in the protease domain. In addition, the polypeptides having BACE activity and comprising a mutation of a noncysteine residue to a cysteine may or may not further comprise an engineered cleavage site.

Amino acid insertions, substitutions, or deletions used to install a mutation to cysteine or an engineered cleavage site into a polypeptide having BACE activity may be introduced using several techniques. Such techniques include, for example, site-directed mutagenesis of the nucleic acid sequence encoding a polypeptide having BACE activity such that the nucleic acid sequence encodes a polypeptide having BACE activity, and further comprising a mutation to cysteine, an engineered cleavage site, or both. Particularly preferred is site-directed mutagenesis using polymerase chain reaction (PCR) amplification [see, for example, U.S. Pat. No. 4,683,195 issued 28 Jul. 1987; and Current Protocols In Molecular Biology, Chapter 15 (Ausubel, F. M., et al., ed., (1991)]. Other site-directed mutagenesis techniques are also well known in the art and are described, for example, in the following publications: Ausubel, F. M., et al., supra, Chapter 8; Molecular Cloning: A Laboratory Manual, 2nd edition (Sambrook, J., et al., 1989); Zoller, M. J. & Smith, M., Methods Enzymol. 100:468-500 (1983); Zoller, M. J. & Smith, M., DNA 3:479-488 (1984); Zoller, M. J. & Smith, M., Nucleic Acids Res., 10:6487-6500 (1982); Brake, A. J., et al., Proc. Natl. Acad. Sci. USA 81:4642-4646 (1984); Botstein, D., et al., Science 229:1193-1201(1985); Kunkel. T. A., et al., Methods Enzymol. 154:367-382 (1987), Adelman, J. P., et al., DNA 2:183-193 (1983); and Carter, P., et al., Nucleic Acids Res. 13:4431-43 (1985).

Amino acid sequence mutants with more than one amino acid substitution located close together in the polypeptide chain may be generated simultaneously, using one oligonucleotide that codes for all of the desired amino acid substitutions. Cassette mutagenesis [Wells, J. A., et al., Gene, 34:315-323 (1985)], and restriction selection mutagenesis [Wells, et al., Philos. Trans. R. Soc. London SerA, 317:415 (1986)] may also be used. If, however, the amino acids are located some distance from one another (e.g. separated by more than ten amino acids), it is more difficult to generate a single oligonucleotide that encodes all of the desired changes. Instead, one of two alternative methods may be employed. In the first method, a separate oligonucleotide is generated for each amino acid to be substituted. The oligonucleotides are then annealed to the single-stranded template DNA simultaneously, and the second strand of DNA that is synthesized from the template will encode all of the desired amino acid substitutions. The alternative method involves two or more rounds of mutagenesis to produce the desired mutant.

Another aspect of the invention is a nucleic acid used to encode the polypeptides having BACE activity and having an engineered cleavage site. The design of these nucleic acids is influenced by the choice of host cell, as mentioned above. It is well known in the art that as a consequence of the degenerate genetic code, different organisms can have distinct preferences of codon usage. Nucleic acids of the invention encoding the polypeptide are preferably optimized for translation of the encoded polypeptide in the particular host chosen, using information known in the art about codon preferences for the host organism. The codon usage preferences of hundreds of species have been catalogued [see www.kazusa.orjp/codon/; Nakamura, Y., et al., Nucl. Acids Res. 28: 292 (2000)]. For example, E. coli codon usage preferences have been particularly well characterized [Ikemura, T., J. Mol. Biol. 151:389-409 (1981); Blake, R. D. & Hinds, P. W., J Biomol. Struct. Dynam. 2:593-606 (1984); Hernan, R. A., et al., Biochemistry 31:8619-8627 (1992)]. Optimal polypeptide expression in E. coli, can be accomplished by the use of silent codon substitution mutagenesis to replace codons used more frequently for expression in human cells with codons used more frequently for expression in E. coli. One embodiment of the invention is a nucleic acid encoding a polypeptide comprising in order from N-terminus to C-terminus:
a) a prodomain comprising at least six contiguous amino acids of SEQ ID NO:3;
b) an engineered cleavage site; and
c) a protease domain;
   wherein the polypeptide is capable of being cleaved at the engineered cleavage site thereby releasing a free protease domain that has BACE activity.
a) Another aspect of the invention is a vector for expressing the polypeptides having BACE activity and having an engineered cleavage site. These vectors comprise a nucleic acid encoding the polypeptide, operably linked to an expression control sequence. Expression and cloning vectors are well known in the art and contain nucleic acid sequences that enable the vector to replicate in one or more selected host cells. Expression vectors including the expression control sequences, e.g., promoters, to be used in the creation of the constructs are preferably optimized for use in the particular host cell chosen. There are numerous commercially available expression vectors for production of protein in a variety of cell types. The vector components generally include, but are not limited to, one or more of the following: a signal sequence, an origin of replication, one or more marker genes, an enhancer element, a promoter, and a transcription termination sequence. Furthermore, Shine-Dalgarno consensus sequences may be employed preceding the start codon to increase translation efficiency of a eukaryotic gene product in a prokaryotic host cell. A particular embodiment is a vector for expressing a polypeptide comprising in order from N-terminus to C-terminus:
b) a prodomain comprising at least six contiguous amino acids of SEQ ID NO:3;
c) an engineered cleavage site; and
d) a protease domain;
   wherein the polypeptide is capable of being cleaved at the engineered cleavage site thereby releasing a free protease domain that has BACE activity. A suitable vector is described in Example 1.
e) Another aspect of the invention is a host cell expressing a polypeptide having BACE activity and having an engineered cleavage site. Host cells are transformed with the expression or cloning vector encoding the polypeptide, and are cultured in conventional nutrient media modified as appropriate for inducing promoters, selecting transformants, or amplifying the genes encoding the desired sequences. In one embodiment, the host cell expresses a polypeptide having BACE activity and comprising an autoproteolysis site. In another embodiment the host cell expresses a polypeptide having BACE activity and comprising an exogenous protease cleavage site. Preferably, the polypeptide comprising an exogenous protease cleavage site is expressed in host cells not natively expressing the corresponding exogenous protease, because isolating the polypeptide comprising an intact exogenous protease cleavage site allows processing to occur under more controlled, in vitro conditions. If the polypeptide having BACE activity is to be used for structural studies, it preferably is expressed in prokaryotic cells, more preferably in bacterial cells, and most preferably, in E. coli cells. Expressing the polypeptides in these cell types avoids the heterogeneous glycosylation observed in polypeptides isolated from eukaryotic systems. In a particular embodiment, the host cell expresses a polypeptide comprising in order from N-terminus to C-terminus:
a) a prodomain comprising at least six contiguous amino acids of SEQ ID NO:3;
b) an engineered cleavage site; and
c) a protease domain;
   wherein the polypeptide is capable of being cleaved at the engineered cleavage site thereby releasing a free protease domain that has BACE activity. Example 2 describes the purification from E. coli of polypeptides having BACE activity and having an autoproteolysis site.

### EXAMPLES

### Example 1

### Construction of Bacterial Expression Plasmids Encoding Polypeptides with BACE Activity and with Engineered Cleavage Sites

In brief, a pRSETC (Novagen)-based E. coli expression plasmid, pB22, encoding human proBACE1 starting at amino acid 22 from the wild-type N-terminus, was used as a template for introduction of mutations by Kunkel mutagenesis [Kunkel, T. A., et al., Methods Enzymol. 154:367-382 (1987)]. The sequence encoding the prodomain and linker had been previously optimized for E. coli expression by silent codon substitution mutagenesis.

Cloning of Soluble Human BACE1

A soluble proprotease gene sequence (bases 64-1362, corresponding to amino acid residues 22-454 of SEQ ID NO:1) was subcloned from pFBHT into the E. coli expression vector pRSETC by PCR, to create pB22, which served as a template for mutagenesis. pFBHT is a modified pFastBac1 plasmid (Gibco/BRL) containing the sequence for TEV protease followed by a (His)₆ tag and a stop signal between the XhoI and HinDIII sites. The subcloning was accomplished as follows. The cDNA encoding full-length human BACE1, bases 1-1551, starting from the initiator Met codon and including an extra 48 bases of mRNA transcript following the stop codon [Vassar, R., et al., Science 286:735-741 (1999)] was obtained by a combination of PCR cloning of the 3′ 1425 bases from human cDNA libraries, and synthesis of the remaining 5′ 126 bases by serial overlapping PCR. All PCR reactions were performed using Advantage2 polymerase (Clontech) according to manufacturer's instructions. A fragment spanning bases 126-374 was obtained by PCR from a human cerebral cortex library and SEQ ID NO:26 and SEQ ID NO:27; a fragment spanning bases 339-770 was obtained by PCR from a Stratagene Unizap XR human brain cDNA library, and SEQ ID NO:28 and SEQ ID NO:29; and the 3′ end fragment, spanning bases 735-1551, was obtained by PCR from a human brain library, using SEQ ID NO:30 and SEQ ID NO:31. The three fragments, having 35 bp of overlap at the junctions, were gel purified and combined in one PCR reaction, using primers to the ends (SEQ ID NO:26 and SEQ ID NO:32) to amplify the 126-1551 product.

| | | |
|---|---|---|
| For2 | GCTGCCCCGGGAGACCGACGAAGA | SEQ ID NO:26 |
| midRev2 | CGGAGGTCCCGGTATGTGCTGGAC | SEQ ID NO:27 |
| midFor | CCAGAGGCAGCTGTCCAGCACATA | SEQ ID NO:28 |
| midRev1 | TCCCGCCGGATGGGTGTATACCAG | SEQ ID NO:29 |
| BACE14 | GTACACAGGCAGTCTCTGGTATACACC | SEQ ID NO:30 |
| BACE11 | GTGTGGTCCAGGGGAATCTCTATCTTCTG | SEQ ID NO:31 |
| BACE5 | GTCATCGTCTCGAGTCACTTCAGCAGGGA | SEQ ID NO:32 |
| | GATGTCATCAG | |

The 126-1551 piece, and the subsequent elongated products, were used as a templates for serial overlapping PCR reactions, to add the remaining 5′-126 bases using SEQ ID NO:33, SEQ ID NO:34 and SEQ ID NO:35 as forward primers, with SEQ ID NO:37 always at the reverse primer.

SEQ ID NO:33

BACE fill2
CGGCTGCCCCTGCGCAGCGGCCTGGGGGGCGCCCCCCTGGGGCTGCGGCT
GCCCCGGGAG

SEQ ID NO:34

BACE fill1
ATGGGCGCGGGAGTGCTGCCTGCCCACGGCACCCAGCACGGCATCCGGCT
GCCCCTGCGC

SEQ ID NO:35

BACE for-EcoRI
CCGGAATTCATGGCCCAAGCCCTGCCCTGGCTCCTGCTGTGGATGGGCGC
GGGAGTG

SEQ ID NO:35 and SEQ ID NO:32 contained EcoRI and XhoI restriction sites, respectively, and digestion of the PCR product, along with the Baculovirus expression vector, pFBHT, with the same enzymes was followed by gel purification and ligation of the resulting DNA fragments, yielding the construct, pFBHT-BACE. This construct was used as a template for PCR amplification of bases 1-1362, corresponding to the preproBACE soluble protease, using SEQ ID NO:36 and SEQ ID NO:37.

SEQ ID NO:36

proFor-Nde CGCCATATGGCGGGAGTGCTGCCTGCCCACGGC

SEQ ID NO:37

PACErev-RI CCGGAATTCTCAGGTTGACTCATCTGTCTGTGGAAT

SEQ ID NO:36 and SEQ ID NO:37 contained NdeI and EcoRI restriction sites, respectively, and digestion of the PCR product, along with the E. coli expression vector, pRSETC, with the same enzymes was followed by gel purification and ligation of the resulting DNA fragments leading to the construct pB1. Vector pB1 was then used as a template for Kunkel mutagenesis [Kunkel, T. A., et al., Methods Enzymol. 154:367-382 (1987)] to delete the BACE1 presequence (bases 1-63), producing the construct pB22. pB22 served as a template for mutagenesis to incorporate the engineered cleavage sites, using the Kunkel method.

Introduction of Engineered Cleavage Sites

Mutations and the oligonucleotides used to introduce them are as follows. For the construct encoding a polypeptide of SEQ ID NO:38, the thrombin cleavage site LVPRGS (SEQ ID NO:4) was inserted between residues 45 and 46 of the aforementioned soluble proBACE1, numbered according to the preprosequence in SEQ ID NO:1. The residues correspond to residues 24 and 31, respectively, of SEQ ID NO:38. The oligonucleotide used for this insertion was SEQ ID NO:39.

| | |
|---|---|
| TQHGIRLPLR SGLGGAPLGL RLPRLVPRGS ETDEEPEEPG RRGSFVEMVD | SEQ ID NO:38 |
| NLRGKSGQGY YVEMTVGSPP QTLNILVDTG SSNEAVGAAP HPFLHRYYQR | |
| QLSSTYRDLR KGVYVPYTQG KWEGELGTDL VSIPHGPNVT VRANIAAITE | |
| SDKFFINGSN WEGILGLAYA EIARPDDSLE PFEDSLVKQT HVPNLPSLQL | |
| CGAGFPLNQS EVLASVGGSM TIGGIDHSLY TGSLWYTPIR REWYYEVIIV | |
| RVEINGQDLK MDCKEYNYDK SIVDSGTTNL RLPKKVFEAA VKSIKAASST | |
| EKFPDGFWLG EQLVCWQAGT TPWNIFPVIS LYLMGEVTNQ SFRITILPQQ | |
| YTRPVEDVAT SQDDCYKFAI SQSSTGTVMG AVIMEGFYVV FDRARKRIGF | |
| AVSACHVHDE FRTAAVEGPF VTLDMEDCGY NTPQTDEST | |
| thrombin | |
| site insert | |
| CTCTTCGTCCGTCTCAGAACCACGCGGAACCAGACGTGGCAGACGCAG | SEQ ID NO:39 |

Another construct encodes for a polypeptide of SEQ ID NO:40 containing the sequence RLPLETD (SEQ ID NO:41). The sequence was created by a single point mutation to a Leu of residue Arg45 of proBACE1, numbered relative to the preprosequence. The mutated residue corresponds to residue 24 of SEQ ID NO:40. The oligonucleotide used to make the amino acid substitution was SEQ ID NO:42.

| | |
|---|---|
| TQHGIRLPLR SGLGGAPLGL RLPLETDEEP EEPGRRGSFV EMVDNLRGKS | SEQ ID NO:40 |
| GQGYYVEMTV GSPPQTLNIL VDTGSSNFAV GAAPHPFLHR YYQRQLSSTY | |
| RDLRKGVYVP YTQGKWEGEL GTDLVSIPHG PNVTVRANIA AITESDKFFI | |
| NGSNWEGILG LAYAEIARPD DSLEPFFDSL VKQTHVPNLF SLQLCGAGFP | |
| LNQSEVLASV GGSMTIGGID HSLYTGSLWY TPIRREWYYE VIIVRVETNG | |
| QDLKMDCKEY NYDKSIVDSG TTNLRLPKKV FEAAVKSIKA ASSTEKFPDG | |
| EWLGEQLVCW QAGTTPWNIE PVISLYLMGE VTNQSFRITI LPQQYLRPVE | |
| DVATSQDDCY KEAISQSSTG TVMGAVIMEG FYVVFDRARK RIGFAVSACH | |
| VHDEFRTAAV EGPFVTLDME DCGYNIPQTD EST | |
| RLPL site CTCTTCGTCGGTCTCCAGTGGCAGACGCAGACCCAGTGGAGC | SEQ ID NO:42 |

Another construct encodes for a polypeptide of SEQ ID NO:43 containing the autoproteolysis site ELNLETD (SEQ ID NO:18). The autoproteolysis site was produced by point mutations of three amino acid residues of proBACE1 —Arg42, Pro44, and Arg45-numbered relative to the preprosequence. The residues were mutated to a Glu, an Asp, and a Leu, respectively, which correspond to residues 21, 23, and 24 of SEQ ID NO:43. The oligonucleotide used to make the three point mutations was SEQ ID NO:44.

| | |
|---|---|
| TQHGIRLPLR SGLGGAPLGL ELNLETDEEP EEPGRRGSFV EMVDNLRGKS | SEQ ID NO:43 |
| GQGYYVEMTV GSPPQTLNIL VDTGSSNFAV GAAPHPFLHR YYQRQLSSTY | |
| RDLRKGVYVP YTQGKWEGEL GTDLVSIPHG PNVTVRANIA AITESDKFFI | |
| NGSNWEGILG LAYAEIARPD DSLEPFFDSL VKQTHVPNLF SLQLCGAGFP | |
| LNQSEVLASV GGSMIIGGID HSLYTGSLWY TPIRREWYYE VIIVRVEING | |
| QDLKMDCKEY NYDKSIVDSG TTNLRLPKKV FEAAVKSTKA ASSTEKFPDG | |
| EWLGEQLVCW QAGTTPWNIE PVISLYLMGE VTNQSFRITI LPQQYLRPVE | |
| DVATSQDDCY KFAISQSSTG TVMGAVIMEG EYVVFDRARK RIGFAVSACH | |
| VHDEFRTAAV EGPEVTLDME DCGYNTPQTD EST | |
| ELNL site CTCTTCGTCGGTCTCCAGGTTCAGTTCCAGACCCAGTGGAGC | SEQ ID NO:44 |

A construct encoding a polypeptide with the autoproteolysis site EINLETD (SEQ ID NO:19) was created from the ELNL-BACE1 construct by using the oligonucleotide of SEQ ID NO:45, which replaces the first Leu of the autoproteolysis site of SEQ ID NO:18 with an Ile. The resulting construct encoded for a polypeptide of SEQ ID NO:46.

| | |
|---|---|
| EINL site GGTCTCCAGGTTGATTTCCAGACCCAG | SEQ ID NO:45 |
| TQHGIRLPLR SGLGGAPLGL EINLETDEEP EEPGRRGSFV EMVDNLRGKS | SEQ ID NO:46 |
| GQGYYVEMTV GSPPQTLNIL VDTGSSNFAV GAAPHPELHR YYQRQLSSTY | |
| RDLRKGVYVP YTQGKWEGEL GTDLVSIPHG PNVTVRANIA AITESDKFFI | |
| NGSNWEGILG LAYAEIARPD DSLEPFFDSL VKQTHVPNLF SLQLCGAGFP | |
| LNQSEVLASV GGSMIIGGID HSLYTGSLWY TPIRREWYYE VTIVRVEING | |
| QDLKMDCKEY NYDKSTVDSG TTNLRLPKKV PEAAVKSIKA ASSTEKFPDG | |
| FWLGEQLVCW QAGTTPWNIF PVTSLYLMGE VTNQSFRITI LPQQYLRPVE | |
| DVATSQDDCY KFAISQSSTG TVMGAVIMEG FYVVFDRARK RIGFAVSACH | |
| VHDEFRTAAV EGPFVTLDME DCGYNIPQTD EST | |

Other constructs having engineered cleavage sites were cloned from the BACE WT plasmid and derivatives thereof using the QuikChange XL Site-Directed Mutagenesis Kit (Stratagene) following the manufacturer's protocol. The first round of mutagenesis converted BACE WT to AP-1, which encodes the polypeptide provided in SEQ ID NO:47, using sense (SEQ ID NO:48) and antisense (SEQ ID NO:49) oligonucleotides.

| | |
|---|---|
| TQHGIRLPLR SGLGGAPLGL RLPRETDEEP EEPEINFSFV EMVDNLRGKS | SEQ ID NO:47 |
| CQGYYVEMTV GSPPQTLNIL VDTGSSNFAV GAAPHPELHR YYQRQLSSTY | |
| RDLRKGVYVP YTQGKWEGEL GTDLVSIPHG PNVTVRANIA AITESDKFFI | |
| NGSNWEGILG LAYAEIARPD DSLEPFFDSL VKQTHVPNLF SLQLCGAGFP | |
| LNQSEVLASV GGSMITGGID HSLYTGSLWY TPIRREWYYE VIIVRVEING | |
| QDLKMDCKEY NYDKSIVDSG TTNLRLPKKV FEAAVKSIKA ASSTEKFPDG | |
| FWLGEQLVCW QAGTTPWNIF PVTSLYLMGE VTNQSFRITI LPQQYLRPVE | |
| DVATSQDDCY KFAISQSSTG TVMGAVIMEG EYVVFDRARK RIGFAVSACH | |
| VHDEFRTAAV EGPEVTLDME DCGYNIPQTD EST | |
| GACGAAGAGCCCGAGGAGCCCGAAATCAACTTCAGCTTTGTGGAGATGGTG | SEQ ID NO:48 |
| CACCATCTCCACAAAGCTGAAGTTGATTTCGGGCTCCTCGGGCTCTTCGTC | SEQ ID NO:49 |

AP-1 was converted to AP-2 using sense and antisense oligonucleotides corresponding to SEQ ID NO:50 and SEQ ID NO:51, respectively. Likewise, AP-1 was converted to AP-3 using sense and antisense oligonucleotides corresponding to SEQ ID NO:52 and SEQ ID NO:53, respectively. The resulting encoded proteins for the AP-2 and AP-3 correspond to SEQ ID NO:54 and SEQ ID NO:55, respectively.

| | |
|---|---|
| GAGCCCGAAATCAACTTCCAGTTTGTGGACATGGTGGACAACCTG | SEQ ID NO:50 |
| CAGGTTGTCCACCATGTCCACAAACTGGAAGTTGATTTCGGGCTC | SEQ ID NO:51 |
| CCCGAGGAGCCCGAAATCAACTTCTCCGCTAGCTTTGTGGAGATG | SEQ ID NO:52 |
| CATCTCCACAAAGCTAGCGGAGAAGTTGATTTCGGGCTCCTCGGG | SEQ ID NO:53 |
| TQHGIRLPLR SGLGGAPLGL RLPPETDEEP EEPEINFQFV DMVDNLRGKS | SEQ ID NO:54 |
| GQGYYVEMTV GSPPQTLNIL VDTGSSNFAV GAAPHPFLHR YYQRQLSSTY | |
| RDLRKGVYVP YTQGKWEGEL GTDLVSIPHG PNVTVRANIA AITESDKFFI | |
| NGSNWEGILG LAYAEIARPD DSLEPFFDSL VKQTHVFNLF SLQLCGAGFP | |
| LNQSEVLASV GGSMIIGGID HSLYTGSLWY TPIRREWYYE VITVRVETNG | |
| QDLKMDCKEY NYDKSIVDSG TTNLRLPKKV FEAAVKSIKA ASSTEKFPDG | |
| FWLGEQLVCW QAGTTPWNIF PVISLYLMGE VTNQSFRITI LPQQYLRPVE | |
| DVATSQDDCY KFAISQSSTG TVMGAVIMEG FYVVFDRARK RIGFAVSACH | |
| VHDEFRTAAV EGPFVTLDME DCGYNIPQTD EST | |
| TQHGIRLPLR SGLGGAPLGL RLPRETDEEP EEPEINFSAS EVEMVDNLRG | SEQ ID NO:55 |
| KSGQGYYVEM TVGSPPQTLN ILVDTGSSNF AVGAAPHPFL HRYYQRQLSS | |
| TYRDLRKGVY VPYTQGKWEG ELGTDLVSIP HGPNVTVRAN IAAITESDKF | |
| FINGSNWEGI LGLAYAEIAR PDDSLEPFFD SLVKQTHVPN LFSLQLCGAG | |
| FPLNQSEVLA SVGGSMTIGG IDHSLYTGSL WYTPIRREWY YEVIIVRVEI | |
| NGQDLKMDCK EYNYDKSIVD SGTTNLRLPK KVFEAAVKSI KAASSTEKFP | |
| DGFWLGEQLV CWQAGTTPWN IFPVISLYLM GEVTNQSFRI TILPQQYLRP | |
| VEDVATSQDD CYKFAISQSS TGTVMGAVIM EGFYVVFDRA RKRIGFAVSA | |
| CHVHDEFRTA AVEGPFVTLD MEDCGYNIPQ TDEST | |

Subsequently, the C-terminal eight residues were removed from AP-1, AP-2, AP-3, AP-4, AP-5 and AP-6 constructs by use of sense (SEQ ID NO:61) and antisense (SEQ ID NO:62) oligonucleotides. The removed residues correspond to residues 447-454 of SEQ ID NO:1. These oligonucleotides were also used to produce a construct encoding a polypeptide corresponding to residues 22-446 of SEQ ID NO:1.

| SEQ ID NO:61 | |
|---|---|
| | ATGGAAGACTGTGGCTACAACTGACCACAGACAGATGAGTCAACC |
| SEQ ID NO:62 | |
| | GGTTGACTCATCTGTCTGTGGTCAGTTGTAGCCACAGTCTTCCAT |

### Example 2

### BACE Protein Preparation Using ELNL-BACE1 and EINL-BACE1 Constructs

The expression plasmid encoding the polypeptide ELNL-BACE1 (SEQ ID NO:43) or EINL-BACE1 (SEQ ID NO:47) was transformed into BL21star (DE3) pLysS E. coli (Invitrogen) and plated onto an LB/amp plate containing 100 μg/mL ampicillin. A single colony from the plate was used to inoculate a 5 mL culture of 2×YT broth containing 50 μg/mL carbenicillin. The culture was grown at 37° C. until the OD₆₀₀ reached between 0.4. and 0.6. Glycerol was added to 15% by volume and the resulting glycerol stocks were frozen in 500 μL aliquots in liquid nitrogen. Aliquots were stored at −80° C. Large-scale expression cultures were started by scraping the glycerol stock into 400 mL 2YT+50 μg/mL carbenicillin. Following overnight growth at 37° C., 50 mL of the culture was used to inoculate 1.5 L of the same media, and after growth at 37° C. to an OD at 600 nm of between 0.7 and 0.8, IPTG was added to a final concentration of 1.0 mM and the induced cultures were grown at 4 h at 37° C.

Cells were harvested by centrifugation at 4K rpm. Cell pellets were resuspended in 100 mL buffer TE (10 mM Tris-HCl, 1 mM EDTA pH 8.0) and lysed using a microfluidizer. The crude extract, containing the protein as insoluble inclusion bodies, was centrifuged at 10 K rpm for 10 min, and the resulting pellet washed by resuspension in PBST (1X phosphate buffered saline pH 7.4 [10 mM sodium phosphate, 150 mM NaCl] with 0.5% TritonX-100) followed by centrifugation at 10 K rpm for 10 min.

Washed inclusion body pellets were solubilized in a urea solution (50 mM CAPS pH 10, 8 M urea, 1 mM EDTA, and 100 mM β-mercaptoethanol) with light agitation or rocking for at least 30 min at room temperature, and remaining insoluble debris was removed by centrifugation in a SS-34 rotor at 18 K rpm for 45 min. The supernatant, which contains denatured IBs, was removed and its absorbance at 280 nm was measured by spectrophotometry. The supernatant was diluted by addition of the urea solution until the OD₂₈₀ absorbance measured between 10 and 12.

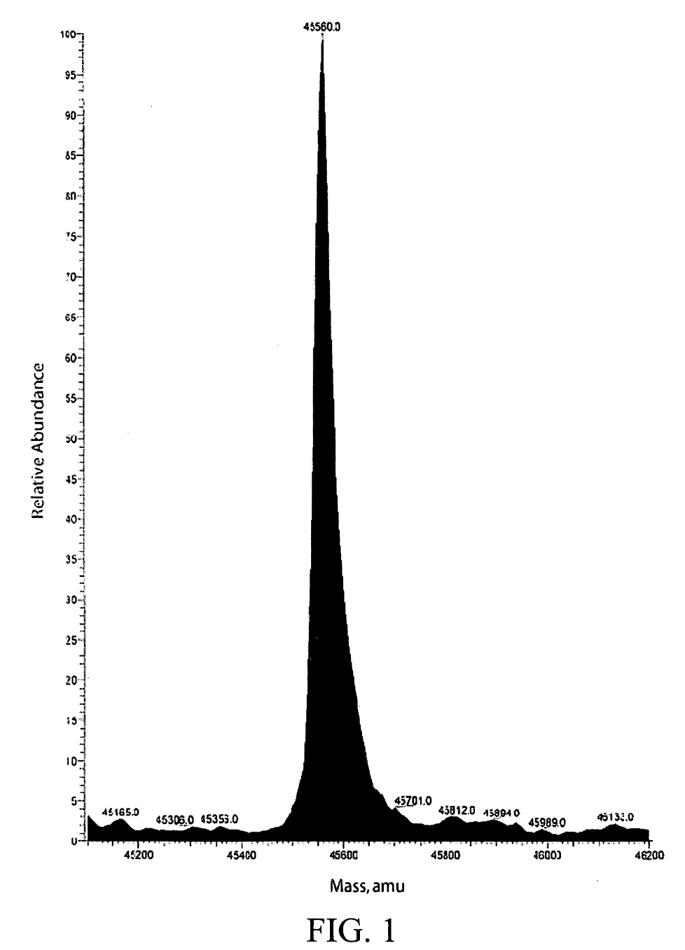

Urea-solubilized ELNL-BACE1 or EINL-BACE1 was refolded by rapid dilution into 50 volumes of rapidly stirred 5 mM Na2CO3, pH 10, followed by incubation at room temperature for 3-5 days. When BACE1 enzymatic activity no longer increased over time, the protein was concentrated 10-20 fold using an ultrafiltration/diafiltration system (Cole-Parmer) and buffer exchanged or dialyzed into 4 mM Tris, pH 8.0, 50 mM NaCl and loaded onto a Q-Sepharose column. Protein was eluted using a linear gradient of 0 to 0.5 M NaCl in 4 mM Tris-HCl pH 8.0 over 8 column volumes. At this point, for the ELNL-BACE1 construct, mass spectral analysis indicated processing to yield an N-terminus starting at residue 42, numbered relative to the preprosequence (corresponding to residue 21 of SEQ ID NO:43). ELNL-BACE1 and EINL-BACE1 were further purified by S-Sepharose chromatography at pH 4.5. Mass spectral analysis for each of the constructs after the S-Sepharose chromatography indicated processing had occurred to yield an N-terminus starting at residue 46, numbered relative to the preprosequence (corresponding to residue 25 of SEQ ID NO:43 and residue 25 of SEQ ID NO:46). The mass spectrum for the processed product of EINL-BACE1 is shown in FIG. 1.

Subsequently, the purified enzyme was dialyzed versus 20 mM Tris pH 7.5 at 4° C. Protein concentrations were determined by absorbance at 280 nm, using ε₂₈₀^{1%}=(0.74). Extinction coefficients were calculated using the Vector NTI software (Informax, Invitrogen Inc.). If protein is to be frozen, glycerol should be added to 15% by volume.

BACE 1 polypeptides were also obtained from the constructs AP-1-AP-6 the cloning of which is described in Example 1. Purification was achieved as for the ELNL construct, except that the S-Sepharose column purification step was not necessary. After acidification and dialysis, the processed BACE1 polypeptides were subjected to mass spectrometry. Calculated and observed masses for the processed certain constructs are shown in Table 1. The observed molecular weight of the construct containing a sequence corresponding to residues 22-446 (preceded by a methionine) is given for reference.

TABLE 1

| | Expected Molecular | Observed Molecular |
|---|---|---|
| Construct | Weight (Da) | Weight (Da) |
| AP-1 | 44083 | 44072 |
| AP-2 | 44110 | 44104 |
| AP-3 | 44237 | 44231 |
| AP-4 | 44083 | 44079 |
| 22-446 | 47329 | 47315 |

### Example 3

### Construction of Bacterial Expression Plasmids Encoding BACE1 Polypeptides with Mutations to Cysteine

The following mutations were introduced by Kunkel mutagenesis into the ELNL-BACE1 construct, the cloning of which is described in Example 1. All residues are numbered relative to preprosequence of human BACE1 (SEQ ID NO:1).

Oligonucleotides for BACE Cysteine Mutations

| | | |
|---|---|---|
| L91C | GCCTGTATCCACACAGATGTTGAGCGT | SEQ ID NO:63 |
| D93C | ACTGCTGCCTGTACACACCAGGATGTT | SEQ ID NO:64 |
| T133C | CCACTTGCCCTGACAGTAGGGCACATA | SEQ ID NO:65 |
| Q134C | TTCCCACTTGCCACAGGTGTAGGGCAC | SEQ ID NO:66 |
| K168C | GTTGATGAAGAAACAGTCTGATTCAGT | SEQ ID NO:67 |
| F169C | GCCGTTGATGAAACACTTGTCTGATTC | SEQ ID NO:68 |
| I171C | GTTGGAGCCGTTACAGAAGAACTTGTC | SEQ ID NO:69 |
| W176C | CAGGATGCCTTCACAGTTGGAGCCGTT | SEQ ID NO:70 |
| I179C | GGCCAGCCCCAGACAGCCTTCCCAGTT | SEQ ID NO:71 |
| I187C | GTCAGGCCTGGCACACTCAGCATAGGC | SEQ ID NO:72 |
| R189C | GGAGTCGTCAGGACAGGCAATCTCAGC | SEQ ID NO:73 |
| Y259C | GATCACCTCATAACACCACTCCCGCCG | SEQ ID NO:74 |
| K285C | GTCCACAATGCTACAGTCATAGTTGTA | SEQ ID NO:75 |
| I287C | GCCACTGTCCACACAGCTCTTGTCATA | SEQ ID NO:76 |
| D289C | GGTGGTGCCACTACACACAATGCTCTT | SEQ ID NO:77 |
| T292C | ACGAAGGTTGGTACAGCCACTGTCCAC | SEQ ID NO:78 |
| N294C | GGGCAAACGAAGACAGGTGGTGCCACT | SEQ ID NO:79 |
| R296C | TTTCTTGGGCAAACAAAGGTTGGTGGT | SEQ ID NO:80 |
| G325C | CACCAGCTGCTCCACTAGCCAGAAACC | SEQ ID NO:81 |
| R368C | ATCTTCCACTGGACACAGGTATTGCTG | SEQ ID NO:82 |
| K382C | TGAGATGGCAAAACAGTAACAGTCGTC | SEQ ID NO:83 |
| S386C | CGTGGATGACTGACAGATGGCAAACTT | SEQ ID NO:84 |
| T390C | CATAACAGTGCCACAGGATGACTGTGA | SEQ ID NO:85 |
| V393C | CAGCTCCCATACAAGTGCCCGTGGATG | SEQ ID NO:86 |

Also made in the ELNL-BACE1 background were inactive polypeptides comprising catalytic site mutations.

Oligonucleotides for Catalytic Site Knock-outs

| | | |
|---|---|---|
| D90N | ACTGCTGCCTGTGTTCACCAGGATGTT | SEQ ID NO:87 |
| D289N | GGTGGTGCCACTGTTCACAATGCTCTT | SEQ ID NO:88 |

Certain cysteine mutants were introduced into the AP-3 background using the QuikChange XL Site-Directed Mutagenesis Kit (Stratagene) following the manufacturer's protocol. The cloning of AP-3 is described in Example 1. The mutants created followed by the sense and antisense oligonucleotides, respectively, used to create each one are: T292C (SEQ ID NO:89 and SEQ ID NO:90), R296C (SEQ ID NO:91 and SEQ ID NO:92), T390C (SEQ ID NO:93 and SEQ ID NO:94), V393C (SEQ ID NO:95 and SEQ ID NO:96), and 1171C (SEQ ID NO:97 and SEQ ID NO:98).

SEQ ID NO:89

AGCATTGTGGACAGTGGCTGCACCAACCTTCGTTTGCCC

SEQ ID NO:90

GGGCAAACGAAGGTTGGTGCAGCCACTGTCCACAATGCT

SEQ ID NO:91

GACAGTGGCACCACCAACCTTTGCTTGCCCAAGAAAGTGTTTGAAGC

SEQ ID NO:92

GCTTCAAACACTTTCTTGGGCAAGCAAAGGTTGGTGGTGCCACTGTC

SEQ ID NO:93

GCCATCTCACAGTCATCCTGCGGCACTGTTATGGGAGCT

SEQ ID NO:94

AGCTCCCATAACAGTGCCGCAGGATGACTGTGAGATGGC

SEQ ID NO:95

CAGTCATCCACGGGCACTTGCATGGGAGCTGTTATCATG

SEQ ID NO:96

CATGATAACAGCTCCCATGCAAGTGCCCGTGGATGACTG

SEQ ID NO:97

GAATCAGACAAGTTCTTCTGCAACGGCTCCAACTGGGAA

SEQ ID NO:98

TTCCCAGTTGGAGCCGTTGCAGAAGAACTTGTCTGATTC

### Example 4

### BACE1 Cysteine Mutant Protein Preparation from Constructs with Engineered Cleavage Sites

Expression

Plasmids expressing human BACE1 having both an autoproteolysis site and a mutation of a native noncysteine residue to cysteine, were separately transformed into BL21 Star (DE3) pLysS and plated on an LB/amp plate (100 μg/mL ampicillin). The plates were grown overnight, after which a freshly transformed colony was used to inoculate a 5 mL culture of 2YT+50 μg/mL carbenicillin. The culture was grown at 37° C. until the OD₆₀₀ reached between 0.4 and 0.6. Glycerol was added to 15% by volume and the resulting glycerol stocks were frozen in 500 μL aliquots in liquid nitrogen. Aliquots were stored at −80° C.

Large-scale expression cultures were started by scraping the glycerol stock into 400 mL 2YT+50 μg/mL carbenicillin, and growing overnight at 37° C. A 50 mL portion of the overnight culture was used to start at least one 1.5 L culture in a shaker flask, which was grown to an OD₆₀₀ near 0.7 or 0.8. At this point, the culture was induced by adding IPTG to a final concentration of 1 mM. The induced cultures were next grown 4 hr at 37° C., although growing longer is also acceptable. Cells were harvested by centrifugation at 6 K rpm.

Refolding/Purification:

Inclusion Body Prep

Cell pellets were resuspended in a minimal amount of TE pH 8.0 (10 mM Tris-HCl, 1 mM EDTA pH 8.0), and lysed fully using either a sonicator or a microfluidizer. A sample of the whole cell lysate was saved to run on an analytical gel. The whole cell lysate was centrifuged 10 min at 10 K rpm, after which the supernatant was removed. A sample of the soluble TE supernatant was also saved for the gel.

The pellet was resuspended in 200 mL PBST (1× phosphate buffered saline pH 7.4 [10 mM sodium phosphate, 150 mM NaCl] with 0.5% TritonX-100). The resuspension was centrifuged for 10 min at 10 K rpm, and washed again with the PBST. A sample of the PBST wash was saved for the gel.

The inclusion body (IB) pellet was resuspended in a volume of a urea solution (8 M Urea, 50 mM CAPS pH 10, 100 mM BME, 1 mM EDTA) such that the resuspension had a final OD₂₈₀ of 8-12 after the subsequent steps of denaturing the protein in the urea and spinning out the insoluble materials. The volume of resuspension therefore depends on the amount of protein present in the IBs. It is convenient to start with less urea solution, and add more if necessary. The pellet in urea solution was incubated with light agitation or rocking for at least 30 min at room temperature, and then centrifuged in SS-34 rotor, for 45 min at 18 K rpm. The supernatant, which contains denatured IBs, was saved. At this point, the OD was measured, and, if necessary, urea was added to bring the value to the desired OD₂₈₀ range of 8-12. During the denaturation of the protein in the urea solution, the introduced cysteine typically forms an adduct with the BME present in the urea solution. The protein-BME adduct in the supernatant can be frozen at −20° C. for later use.

Refolding

A fresh solution of 0.25 M sodium carbonate pH 10 was used to make up a refolding buffer containing 5 mM Na₂CO₃ pH 10. The protein in the urea solution having an OD₂₈₀ of 8-12 was diluted 1:50 by volume into the refolding buffer by quickly adding the urea solution to the larger volume of 5 mM Na₂CO₃ pH 10, as the larger volume was stirred on a stir-plate. The activity of the BACE protein, i.e. the BACE-BME adduct, was monitored over the next 3-5 d. We have noted the pH of the solution drops to approximately 9.8 after protein addition, and ends at 9.5 over the course of 5 d. The protein in the refolding buffer was concentrated, and its buffer exchanged using an ultrafiltration/diafiltration system (Masterflex L/S, Cole-Parmer). Generally, concentrating 10-20 fold gave excellent results with little to no precipitation in the solution. Exchange of buffer can be accomplished by diafiltration against 2-3 L Q load buffer (4 mM Tris pH 8.0, 50 mM NaCl) or if more convenient, dialyzing against the Q load buffer.

Q Sepharose FF

The protein sample was prepared in the same buffer used to equilibrate and wash the column, that is it was dialyzed into buffer A (4 mM Tris pH 8.0, 50 mM NaCl). For example, if the protein was present in 2 L of refold solution, it was dialyzed twice against 20 L over 7 or 8 h. The protein solution was removed from the dialysis bag and filtered before loading the column.

Before loading, the column was washed first in high salt (1-2 M) until the UV absorbance leveled out and then in the protein loading buffer (Buffer A) until the UV absorbance leveled out. The high salt buffer was usually Buffer B (4 mM Tris pH 8.0, 1 M NaCl), which was subsequently used to elute the protein from the column. This column preparation step can be done before every run or after every run, but is preferably done after the run.

While loading the protein onto the column, the flow rate should not exceed 5 mL/min; with large volumes it is helpful to set up a slow overnight load. As the sample was drawn up into the tubing, it was watched, and stopped just before air was drawn into the tube. The flow through from the waste line is preferably collected during loading, as sometimes the protein does not stick to the column and can be found in the flow through. After the protein was loaded, the column was washed with 4-5 column volumes of Buffer A. The UV absorbance on the chromatogram should level out before starting the elution gradient.

Although there are several types of gradients that may be employed, for BACE cysteine mutant purification on a 20 mL Q seph FF column, the following linear gradient was typically used: 0-75% Buffer B, 4 mL/min over 40 min, collected in 7 mL fractions, and followed by a short 100% Buffer B wash at the end of the gradient. The fractions were collected during the wash step as well. The fractions were checked for refolded protein by running a non-reducing 10% Bis-Tris PAGE with MOPS buffer, and the appropriate fractions were combined and dialyzed overnight into dH₂O.

Acidification

The protein was next buffered by adding 1 M sodium acetate pH 4.5, to a final concentration of 20 mM. In general, the addition is best done quickly while mixing the solution. The resulting solution was left for 5-10 min with stirring and any resulting precipitation was filtered. The pH drop can first be done on a small scale (0.5-1 mL). After ensuring that the appropriate material fell out of solution by gel analysis of the supernatant, the remainder of the preparation was acidified. The acidified protein was filtered first using a 0.45 μm filter, and then using a 0.22 μm filter.

S Sepharose Column

At this point, the protein was characterized by assaying activity, by mass spectrometry and by polyacrylamide gel electrophoresis (PAGE). If purity was satisfactory at this stage, the protein was dialyzed into reducing buffer, as described below. If the purity was unsatisfactory, the protein was further purified on a 5 mL S sepharose fast-flow column. Loading flow rate depends on protein concentration; ˜1 mg protein/min was used in general. The protein was loaded in 10 mM sodium acetate pH 4.5, and next a linear gradient from 0-100% at 2 mL/min, over 20 min was run, with collection of 3 mL fractions. Fractions containing protein were pooled and dialyzed into reducing buffer in a timely manner, because active BACE autoproteolyzes. Before dialyzing, the OD₂₈₀ of the protein was measured, using 0.5 M NaOAc as a blank.

Reduction of Cysteine Mutant Proteins

The purified protein was dialyzed into reducing buffer containing 20 mM Tris pH 7.5, 125 mM NaCl, 1 mM DTT. The protein was checked periodically by mass spectrometry to monitor removal of β-mercaptoethanol (BME) from the cysteine-BME adduct formed during the interaction with the urea solution containing the BME; the removal was effected by a reduction reaction with the DTT. If the BME was slow to reduce, the dialysis buffer DTT concentration was increased to 5 mM. If the BME was particularly slow to reduce, one of the following two procedures was followed. The first procedure was to add 0.4 M urea to the reducing buffer and to continue checking reduction status. The second procedure was to exchange the protein into 20 mM Tris pH 7.5 thereby removing all of the DTT and urea, and then to add TCEP to a final concentration of 2 mM, by direct addition from a 0.5 M TCEP stock. In either case, the reduction status was monitored. If using TCEP or 5 mM DTT, the number of cysteine modifications by cystamine or by a suitably reactive compound was measured to ensure that the native disulfides were not reduced as well. After the protein was reduced, it was dialyzed into storage buffer (20 mM Tris pH 7.5). If protein was to be frozen, glycerol was added to 15% by volume. Protein concentrations were determined by absorbance at 280 nm, using an ε₂₈₀ as calculated using Vector NTI software (Informax, Invitrogen Inc.); typical ε₂₈₀ for cysteine mutants range from 0.70 to 0.72.

What is claimed is:

1-24. (canceled)

25. A nucleic acid encoding the polypeptide comprising in order from N-terminus to C-terminus:
(i) a prodomain comprising at least six contiguous amino acids of SEQ ID NO:3;
(ii) an autoproteolysis site comprising the sequence X₁X₂X₃X₄X₅,
wherein
X₁ is Glu or Gln.
X₂ is Leu, Ile or Val,
X₃ is Asn, Asp or Met,
X₄ is Leu or Phe, and
X₅ is Glu, Met, Gln, Ser, Ala or Asp; and
(iii) a protease domain comprising at least one amino acid sequence selected from the group consisting of
(a) a sequence at least 90% identical to residues 74-207 of SEQ ID NO:1,
(b) a sequence at least 90% identical to residues 241-361 of SEQ ID NO:1 and
(c) a sequence at least 90% identical to residues 389-446 of SEQ ID NO:1;
wherein the polypeptide is capable of being cleaved at the autoproteolysis site thereby releasing a free protease domain that has BACE activity.

26. A vector for expression of the encoded polypeptide of claim 25.

27. A host cell expressing the encoded polypeptide of claim 25.

28. The nucleic acid of claim 25 wherein the protease domain of the encoded polypeptide comprises at least two sequences selected from (a), (b) and (c).

29. The nucleic acid of claim 28 wherein the protease domain of the encoded polypeptide comprises each of (a), (b) and (c).

30. The nucleic acid of claim 28 wherein the protease domain of the encoded polypeptide comprises at least one sequence selected from the group consisting of a sequence at least 97% identical to residues 74-207 of SEQ ID NO:1; a sequence at least 97% identical to residues 241-361 of SEQ ID NO:1; and a sequence at least 97% identical to residues 389-446 of SEQ ID NO:1.

31. The nucleic acid of claim 28 wherein the encoded polypeptide does not include at least one sequence selected from the group consisting of a sequence corresponding to residues 217-231 of SEQ ID NO:1 and a sequence corresponding to residues 371-379 of SEQ ID NO:1.

32. The nucleic acid of claim 31 wherein the encoded polypeptide does not include a sequence corresponding to residues 217-231 of SEQ ID NO:1 and a sequence corresponding to residues 371-379 of SEQ ID NO:1.

33. The nucleic acid of claim 28 or claim 31 wherein the protease domain of the encoded polypeptide comprises at least one sequence selected from the group consisting of LQLCX₁X₂X₃X₄GGSM (SEQ ID NO:16) and LRPVX₁X₂X₃X₄CYKF (SEQ ID NO:17); wherein each instance of X₁ and X₂ is independently any amino acid and wherein each instance of X₃ and X₄ is independently absent or any amino acid.

34. The nucleic acid of claim 33 wherein the protease domain of the encoded polypeptide comprises both SEQ ID NO:16 and SEQ ID NO:17.

35. The nucleic acid of claim 28 wherein the encoded polypeptide has at its C-terminus a sequence corresponding to residues 447-454 of SEQ ID NO:1.

36. The nucleic acid of claim 28 wherein the encoded polypeptide has at its C-terminus a sequence corresponding to residues 441-443 of SEQ ID NO:1.

37. The nucleic acid of claim 28 wherein the autoproteolysis site of the encoded polypeptide comprises the sequence X₁X₂X₃X₄X₅,
wherein
X₁ is Glu,
X₂ is Leu, Ile or Val,
X₃ is Asn,
X₄ is Leu or Phe, and
X₅ is Glu, Gln, Ser, or Asp.

38. The nucleic acid of claim 28 wherein the autoproteolysis site of the encoded polypeptide is selected from the group consisting of ELNLETD (SEQ ID NO:18), EINLETD (SEQ ID NO:19), EINFETD (SEQ ID NO:20), EVNLDAE (SEQ ID NO:21), EINFSFVE (SEQ ID NO:22), EINFQFVD (SEQ ID NO:23) and EINFSASF (SEQ ID NO:24).

39. A nucleic acid encoding a polypeptide comprising a sequence at least 85% identical to residues 22-446 of SEQ ID NO:1, wherein the polypeptide comprises an autoproteolysis site selected from the group consisting of ELNLETD (SEQ ID NO:18), EINLETD (SEQ ID NO:19), EINFETD (SEQ ID NO:20), EVNLDAE (SEQ ID NO:21), EINFSFVE (SEQ ID NO:22), EINFQFVD (SEQ ID NO:23) and EINFSASF (SEQ ID NO:24).

40. The nucleic acid of claim 39 wherein the encoded polypeptide has at its C-terminus a sequence corresponding to residues 441-446 of SEQ ID NO:1.

41. The nucleic acid of claim 39 wherein the encoded polypeptide has at its C-terminus a sequence corresponding to residues 447-454 of SEQ ID NO:1.
